(19) United States
(12) Patent Application Publication
(10) Pub. No.: US 2024/0279638 A1
(43) Pub. Date: Aug. 22, 2024

(54) MATERIALS AND METHODS FOR PURIFYING DNA, RNA, AND POLYPEPTIDES

(71) Applicant: Impact Proteomics, Pittsburgh, PA (US)

(72) Inventors: Jonathan Minden, Pittsburgh, PA (US); Stephanie Biedka, Pittsburgh, PA (US)

(21) Appl. No.: 18/438,664

(22) Filed: Feb. 12, 2024

Related U.S. Application Data
(60) Provisional application No. 63/486,050, filed on Feb. 21, 2023.

(51) Int. Cl. C12N 15/10; C12N 9/76

(52) U.S. Cl. CPC C12N 15/101; C12N 9/6427; C12N 15/1013

(57) ABSTRACT

Materials and methods for purifying DNA, RNA, and protein from a single sample are provided.

### GOVERNMENT SUPPORT

This invention was made with government support under Contract No. IIP-2036199 awarded by the National Science Foundation. The government has certain rights in the invention.

### FIELD OF USE

The present disclosure relates to materials and methods for purifying DNA, RNA, and polypeptides from a single sample.

### BACKGROUND

A major complication for multi-omics research and diagnostics is sample preparation of DNA, RNA, and protein from a single sample. Typically, three separate samples are processed by three different methods or kits to yield representative DNA, RNA, and protein samples. The distributive approach requires larger initial samples divided into three portions, which introduces heterogeneity. The approach is costly because it requires three kits and is also time consuming because the kits are used independently.

Currently, there are challenges with materials and methods for purifying DNA, RNA, and protein from a sample.

### SUMMARY

Provided herein is a method of purifying DNA, RNA, and polypeptides from a sample. The method comprises: mixing a sample comprising DNA, RNA, and polypeptides with a compound, thereby forming a mixed sample, wherein the compound comprises a first moiety comprising a first member of a bio-orthogonal coupling pair, wherein the first member is configured to form a covalent bond with a second member of a bio-orthogonal coupling pair, a second moiety configured to form a pH dependent covalent bond with a polypeptide, and a linker linking the first moiety and the second moiety; coupling polypeptides in the mixed sample to the second member of the bio-orthogonal coupling pair, wherein the second member of the bio-orthogonal coupling pair is linked to a substrate, thereby forming polypeptides bound to the substrate; precipitating RNA and DNA in the mixed sample using a polar aprotic solvent, thereby forming precipitated RNA and precipitated DNA; resolubilizing and eluting the precipitated RNA; resolubilizing and eluting the precipitated DNA; and eluting the polypeptides bound to the substrate in an elution buffer having a pH more acidic than a pH of the mixed sample by reversing the pH dependent covalent bond between the polypeptides and the second moiety.

Also provided herein is a method of purifying DNA, RNA, and polypeptides from a sample. The method comprises: coupling polypeptides in a mixed sample to a second member of a bio-orthogonal coupling pair, wherein the second member of the bio-orthogonal coupling pair is linked to a substrate, thereby forming polypeptides bound to the substrate, and wherein the mixed sample comprises DNA, RNA, polypeptides, and a compound comprising a first moiety comprising a first member of a bio-orthogonal coupling pair, wherein the first member is configured to form a covalent bond with a second member of a bio-orthogonal coupling pair, a second moiety configured to form a pH dependent covalent bond with a polypeptide, and a linker linking the first moiety and the second moiety; precipitating RNA and DNA in the mixed sample using a polar aprotic solvent, thereby forming precipitated RNA and precipitated DNA; resolubilizing and eluting the precipitated RNA; resolubilizing and eluting the precipitated DNA; and eluting the polypeptides bound to the substrate in an elution buffer having a pH more acidic than a pH of the mixed sample by reversing the pH dependent covalent bond between the polypeptides and the second moiety.

Also provided herein is a method of purifying DNA, RNA, and polypeptides from a sample. The method comprises: mixing a sample comprising DNA, RNA, and polypeptides with a compound, thereby forming a mixed sample, wherein the compound comprises a first moiety comprising a first member of a bio-orthogonal coupling pair, wherein the first member is configured to form a covalent bond with a second member of a bio-orthogonal coupling pair, a second moiety configured to form a covalent bond with a polypeptide, and a linker linking the first moiety and the second moiety; coupling polypeptides in the mixed sample to the second member of the bio-orthogonal coupling pair, wherein the second member of the bio-orthogonal coupling pair is linked to a substrate, thereby forming polypeptides bound to the substrate; precipitating RNA and DNA in the mixed sample using a polar aprotic solvent, thereby forming precipitated RNA and precipitated DNA; resolubilizing and eluting the precipitated RNA; resolubilizing and eluting the precipitated DNA; adding a modified protease to the polypeptides bound to the substrate, thereby forming peptides, wherein the modified protease comprises a protease, and a first member of a bio-orthogonal coupling pair attached to the protease, wherein the first member is configured to form a covalent bond with a second member of a bio-orthogonal coupling pair; coupling the modified protease to a second member of a bio-orthogonal coupling pair, wherein the second member of the bio-orthogonal coupling pair is linked to the substrate; and eluting the peptides.

Also provided herein is a method of purifying DNA, RNA, and polypeptides from a sample. The method comprises: coupling polypeptides in a mixed sample to a second member of a bio-orthogonal coupling pair, wherein the second member of the bio-orthogonal coupling pair is linked to a substrate, thereby forming polypeptides bound to the substrate, wherein the mixed sample comprises DNA, RNA, polypeptides, and a compound comprising a first moiety comprising a first member of a bio-orthogonal coupling pair, wherein the first member is configured to form a covalent bond with a second member of a bio-orthogonal coupling pair, a second moiety configured to form a covalent bond with a polypeptide, and a linker linking the first moiety and the second moiety; precipitating RNA and DNA in the mixed sample using a polar aprotic solvent, thereby forming precipitated RNA and precipitated DNA; resolubilizing and eluting the precipitated RNA; resolubilizing and eluting the precipitated DNA; adding a modified protease to the polypeptides bound to the substrate, thereby forming peptides, wherein the modified protease comprises a protease and a first member of a bio-orthogonal coupling pair attached to the protease, wherein the first member is configured to form a covalent bond with a second member of a bio-orthogonal coupling pair; coupling the modified protease to a second member of a bio-orthogonal coupling pair, wherein the second member of the bio-orthogonal coupling pair is linked to the substrate; and eluting the peptides.

Also provided herein is a multi-omic isolation kit. The multi-omic isolation kit comprises a compound comprising a first moiety comprising a first member of a bio-orthogonal coupling pair, wherein the first moiety is configured to form a covalent bond with a second member of a bio-orthogonal coupling pair, a second moiety configured to form a pH dependent covalent bond with a polypeptide, and a linker linking the first moiety and the second moiety; a substrate linked to a second member of a bio-orthogonal coupling pair, wherein the substrate is used to isolate DNA, RNA, and polypeptides; and a polar aprotic solvent.

Also provided herein is a multi-omic isolation kit. The multi-omic isolation kit comprises a compound comprising a first moiety comprising a first member of a bio-orthogonal coupling pair, wherein the first moiety is configured to form a covalent bond with a second member of a bio-orthogonal coupling pair, a second moiety configured to form a covalent bond with a polypeptide, and a linker linking the first moiety and the second moiety; a substrate linked to a second member of a bio-orthogonal coupling pair, wherein the substrate is used to isolate DNA, RNA, polypeptides, and modified protease; a polar aprotic solvent; a modified protease comprising a protease and a first member of a bio-orthogonal coupling pair attached to the protease, wherein the first member is configured to form a covalent bond with a second member of a bio-orthogonal coupling pair.

It is understood that the inventions disclosed and described in this specification are not limited to the aspects summarized in this Summary. The reader will appreciate the foregoing details, as well as others, upon considering the following detailed description of various non-limiting and non-exhaustive aspects according to this specification.

### BRIEF DESCRIPTION OF THE DRAWINGS

Various aspects of the materials and methods disclosed and described in this specification can be better understood by reference to the accompanying figures, in which:

FIGS. 1A and 1B illustrate the formation of a pH dependent tagged polypeptide. FIG. 1A shows compound 1 comprising a first moiety, a linker, and a second moiety configured to form a pH dependent covalent bond with a polypeptide. FIG. 1B shows compound 1 and a polypeptide forming a pH dependent tagged polypeptide;

FIG. 2 shows an example of compound 1 (e.g. mTet-PEGₙ-CDM) and a polypeptide forming a pH dependent tagged polypeptide (e.g. mTet-PEGₙ-CDM tagged polypeptide);

FIGS. 3A and 3B illustrate the formation of a permanently tagged polypeptide. FIG. 3A shows compound 2 comprising a first moiety, a linker, and a second moiety configured to form a covalent bond with a polypeptide. FIG. 3B shows compound 2 and a polypeptide forming a permanently tagged polypeptide;

FIG. 4 shows an example of compound 2 (e.g. mTet-PEGₙ-NHS) and a polypeptide forming a permanently tagged polypeptide (e.g. mTet-PEGₙ-NHS tagged polypeptide);

FIG. 5 shows a schematic of a non-limiting method of purifying DNA, RNA, and polypeptides from a single sample when using compound 1 for tagging polypeptides within the single sample;

FIG. 6 shows a schematic of a non-limiting method of purifying DNA, RNA, and polypeptides from a single sample when using compound 2 for tagging polypeptides within the single sample;

FIG. 7 shows a schematic depiction of an exemplary cartridge;

FIG. 8A is an SDS-PAGE gel showing the amount of protein (lysozyme, carbonic anhydrase, alcohol dehydrogenase, and yeast lysate) recovered from mTet-PEGₙ-CDM tagged polypeptides coupled to trans-cyclooctene (TCO) beads at various times after lowering the pH to at or near 3.

FIG. 8B is a graph showing the percentage of protein (lysozyme, carbonic anhydrase, alcohol dehydrogenase, and yeast lysate) recovered from mTet-PEGₙ-CDM tagged polypeptides coupled to TCO beads at various times after lowering the pH to at or near 3.

FIG. 9A is a table showing the proteins and peptides purified from yeast cell lysates A, B, and C using a conventional method;

FIG. 9B is a table showing the proteins and peptides purified from yeast cell lysates A, B, and C using the method of purifying DNA, RNA, and polypeptides disclosed herein.

FIG. 9C is an illustration showing the proteins purified from yeast cell lysates A, B, and C using a conventional method;

FIG. 9D is an illustration showing the proteins and peptides purified from yeast cell lysates A, B, and C using the method of purifying DNA, RNA, and polypeptides disclosed herein.

FIG. 10A is a gel electrophoresis showing eluted yeast genomic DNA;

FIG. 10B is a gel electrophoresis showing a 580-base pair product, Rpf2, amplified from yeast genomic DNA during a polymerase chain reaction (PCR).

FIG. 10C is a table showing whole genome sequencing (WGS) results of yeast genomic DNA purified using the method of purifying DNA, RNA, and polypeptides disclosed herein compared to purification using a conventional method, PROMEGA WIZARD® genomic DNA purification kit;

FIG. 11A is a table showing the proteins and peptides purified from human embryonic kidney (HEK) cell lysates A, B, C, and D using the method of purifying DNA, RNA, and polypeptides disclosed herein.

FIG. 11B is an illustration showing the proteins purified from HEK cell lysates A, B, C, and D using the method of purifying DNA, RNA, and polypeptides disclosed herein.

FIG. 12A is a gel electrophoresis showing eluted genomic DNA from HEK cell lysates A, B, C, and D;

FIG. 12B is a table showing WGS results of genomic DNA purified from HEK cell lysates A, B, C, and D using the method of purifying DNA, RNA, and polypeptides disclosed herein;

FIG. 13 is a table showing RNA sequencing results of cancer cell line RNA purified using the method of purifying DNA, RNA, and polypeptides disclosed herein;

FIG. 14 is a table showing WGS results of cancer cell line genomic DNA purified using the method of purifying DNA, RNA, and polypeptides disclosed herein;

FIG. 15 is Principle Component Analysis (PCA) of proteomes of four different cancer cell lines;

### DETAILED DESCRIPTION OF NON-LIMITING EMBODIMENTS

Disclosed herein are methods of purifying DNA, RNA, and polypeptides from a single sample.

In one aspect of the present disclosure, methods of purifying DNA, RNA and polypeptides from a single sample require compounds comprising a first moiety, a linker, and a second moiety.

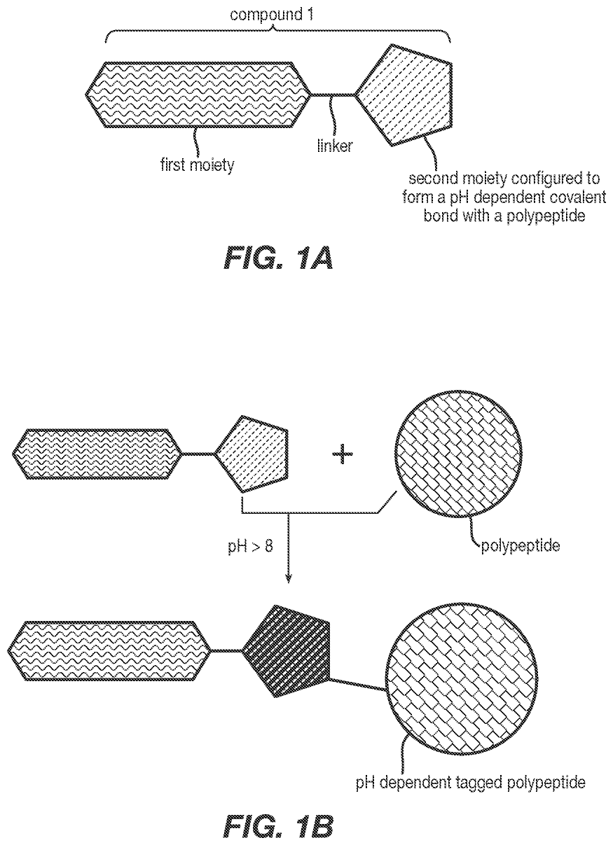

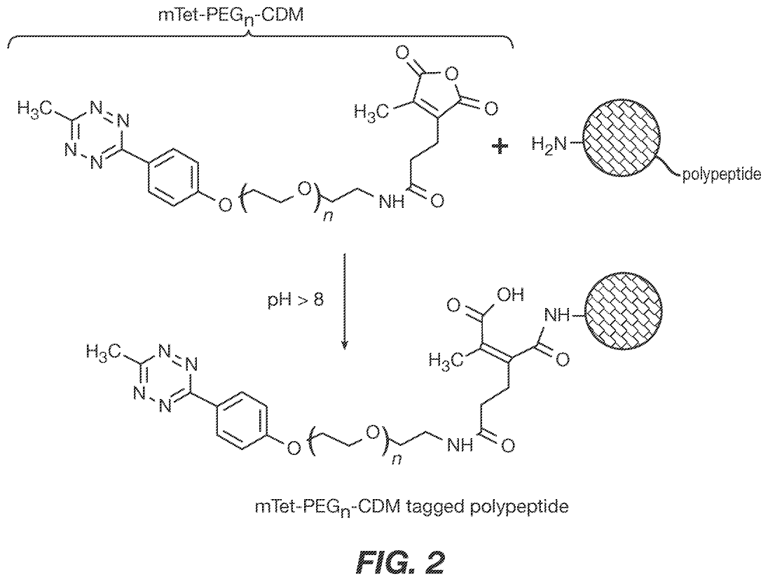

In certain embodiments, the second moiety of the disclosed compounds can be configured to form a pH dependent covalent bond with polypeptides. These compounds are referred to herein as “compound 1” and illustrated in FIG. 1A. Compound 1 can form a pH dependent covalent bond with a polypeptide thereby forming a pH dependent tagged polypeptide, as illustrated in FIG. 1B. In certain embodiments, the second moiety of compound 1 can comprise a dicarboxylic acid anhydride moiety. In certain other embodiments, the dicarboxylic acid anhydride moiety can be a maleic anhydride moiety. In certain other embodiments, the dicarboxylic acid anhydride moiety can be a 2-(2′-carboxyethyl) maleic anhydride moiety. In certain embodiments, the compound can be mTet-PEGₙ-CDM and form a pH dependent covalent bond with a polypeptide thereby forming a mTet-PEGₙ-CDM tagged polypeptide, as illustrated in FIG. 2.

Compound 1 can be bonded to a polypeptide by formation of an amide bond between the dicarboxylic anhydride of the compound and primary amines of the polypeptide. The reaction of the dicarboxylic anhydride moiety with primary amines of a polypeptide can result in the formation of an acid-labile amide bond, which can only be cleaved by mild acids (e.g. acids with a pH ranging from >2 to 6).

An embodiment of Compound 1 is described in WO 2019/236988, which is incorporated herein by reference in its entirety.

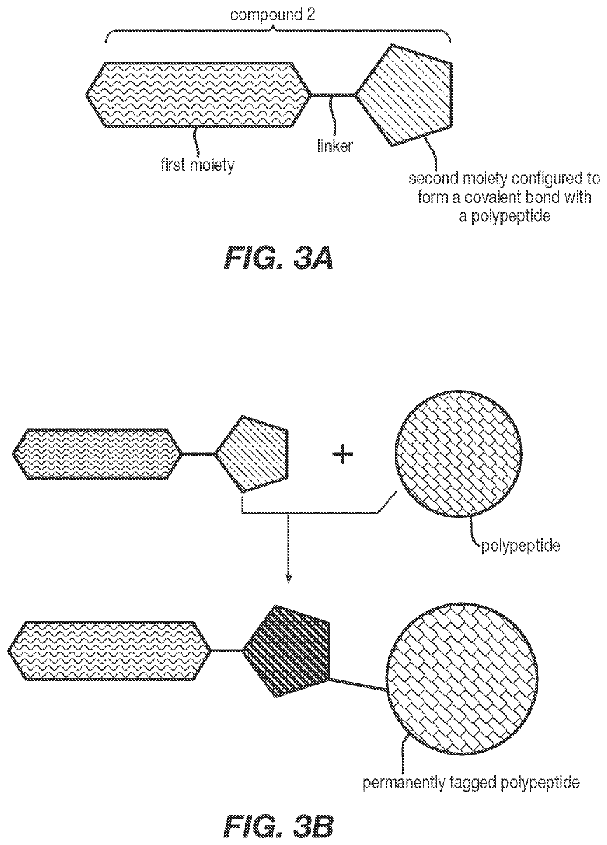

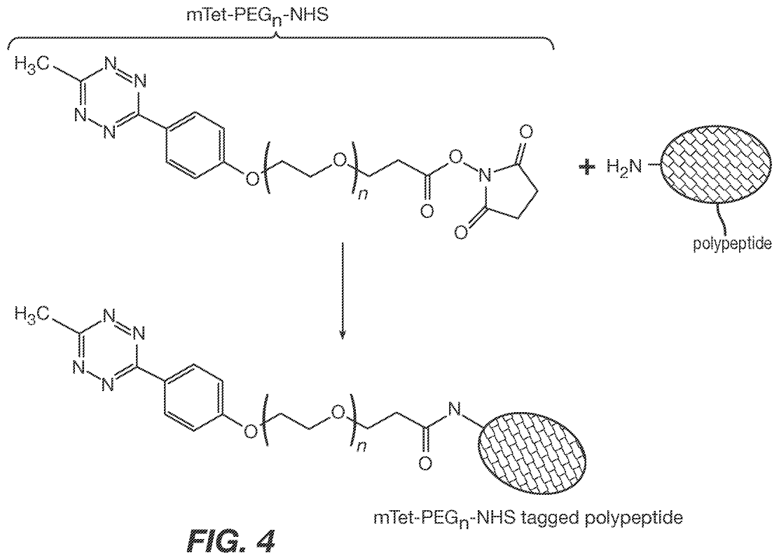

In certain embodiments, the second moiety of the disclosed compounds can be configured to form a covalent bond with polypeptides. These compounds are referred to herein as “compound 2” and illustrated in FIG. 3A. Compound 2 can form a permanent covalent bond with a polypeptide thereby forming a permanently tagged polypeptide, as illustrated in FIG. 3B. In certain embodiments, the second moiety of compound 2 can comprise a N-hydroxysuccinimide moiety. In certain embodiments, the compound can be mTet-PEGₙ-NHS and form a covalent bond with a polypeptide thereby forming a mTet-PEGₙ-NHS tagged polypeptide, as illustrated in FIG. 4.

“Click Chemistry” describes reactions that are high yielding, wide in scope, create only byproducts that can be removed without chromatography, are stereospecific, simple to perform, and can be conducted in easily removable or benign solvents. In the context of the present disclosure a click chemistry reaction is biorthogonal, meaning it is sufficiently selective that it can be performed reliably even in a complex biological environment. These reactions may proceed efficiently in the presence of the multitude of functional groups found in living systems such nucleophiles, electrophiles, reductants, oxidants, and water. Simultaneously, these reactions should have a minimal impact on the biology itself.

In the context of bio-orthogonal reactions, e.g., a bio-orthogonal click chemistry reaction, relies on bond formation between molecules or moieties not found in natural compounds, referred to herein as bio-orthogonal coupling pairs. Such reactions can be selective over other potential reactive functional groups present on biomolecules, proceed in aqueous media at near physiological pH, and have fast reaction rates at room temperature (or up to 37° C.) using low reactant concentrations, which can ensure high modification efficiency (Lopes Bernardes, G., Oliveira, B., & Guo, Z. 2017. Inverse electron demand Diels-Alder reactions in chemical biology. Chemical Society Reviews https://doLorg/10.17863/CAM.10698). Bio-orthogonal reaction reagents (bio-orthogonal coupling pairs) do not react with natural cellular products, such as proteins or nucleic acids. Coupling occurs under a wide range of aqueous conditions and are stable once formed. In one embodiment, the bio-orthogonal reaction can be an inverse-electron-demand Diels-Alder reaction (IEDDA, e.g., inverse electron demand [4+2] Diels-Alder cycloaddition), in which an electron-rich dienophile reacts (e.g., a strained alkene) with an electron-poor diene (e.g., a tetrazine such as a 1,2,4,5-tetrazine or a 4-(1,2,4,5-tetrazinyl)phenyl moiety such as 4-(1,2,4,5-tetrazin-3-yl)phenyl, 6-alkyl-1,2,4,5-tetrazine, 6-pyridin-2-yl-1,2,4,5-tetrazine, 6-pyrimidin-2-yl-1,2,4,5-tetrazine, 4-(6-alkyl-1,2,4,5-tetrazin-3-yl)phenyl, 4-(6-pyridin-2-yl-1,2,4,5-tetrazin-3-yl)phenyl, or 4-(6-pyrimidin-2-yl-1,2,4,5-tetrazin-3-yl)phenyl, where alkyl may be a C₁₋₄ alkyl group) (see, e.g., Karver, M R, et al., Synthesis and Evaluation of a Series of 1,2,4,5-tetrazines for Bio-orthogonal Conjugation. Bioconjug Chem. 2011 Nov. 16; 22(11):2263-2270) in contrast to a normal electron demand Diels-Alder reaction, where an electron-rich diene reacts with an electron-poor dienophile (see, e.g., Lopes Bernardes, G., Oliveira, B., & Guo, Z. 2017. Chemical Society Reviews https://doi.org/10.17863/CAM.10698 for further details, as well as providing examples of other bio-orthogonal reactions). Of note “1,2,4,5-tetrazine” refers to the precise 1,2,4,5-tetrazine compound or a 1,2,4,5-tetrazinyl moiety, while “a 1,2,4,5-tetrazine” refers to a compound or moiety comprising the 1,2,4,5-tetrazinyl moiety. In another example, the bio-orthogonal coupling pair is an alkyne-azide coupling pair, such as a propargyl moiety and an azido moiety, as are broadly-known.

In IEDDA reactions, Electron-poor dienes, such as 1,2,4,5-tetrazines, are reacted with an electron-rich dienophile, for example, a strained dienophile, and fine-tuning the choice of electron-poor diene and electron-rich dienophile (“IEDDA coupling pair”) can be used to tailor the reaction kinetics (Id.). Non-limiting examples of suitable electron-poor dienes for IEDDA reactions can include: tetrazines, such as 1,2,4,5 tetrazines, e.g. methyltetrazine and triazines (see, e.g., Devaraj, N K, et al., Fast and Sensitive Pretargeted Labeling of Cancer Cells via Tetrazine/Trans-Cyclooctene Cycloaddition Agnew Chem Int Ed Engl. 2009; 48(38):7013-7016 and Karver, M R, et al., Bioconjug Chem. 2011 Nov. 16; 22(11):2263-2270). A non-limiting example of an electron-rich dienophile for IEDDA reactions can be trans-cyclooctene. One non-limiting example of a bio-orthogonal reaction and reaction pair can be the reaction of methyltetrazine (mTet) and trans-cyclooctene (TCO). That IEDDA pair was used because of the fast reaction kinetics and the reaction does not need a catalyst. Other suitable bio-orthogonal coupling reactions include alkyne-azide reactions, e.g. with a triazide and an alkyne, or alkyne-DBCO (dibenzocyclooctyne) reactions.

In certain embodiments, compound 1 and compound 2 disclosed herein can comprise a first moiety comprising a first member of a bio-orthogonal coupling pair, wherein the first member is configured to form a covalent bond with a second member of a bio-orthogonal coupling pair.

In certain embodiments, the first member of the bio-orthogonal coupling pair can be an electron-poor diene, an electron-rich dienophile, or a strained cycloalkene. In certain other embodiments, the first member of a bio-orthogonal coupling pair can be a tetrazine moiety selected from the group consisting of a 1,2,4,5-tetrazine moiety and a 4-(1,2,4,5-tetrazinyl)phenyl moiety.

In certain embodiments, the second member of the bio-orthogonal coupling pair can be an electron-poor diene, an electron-rich dienophile, or a strained cycloalkene. In certain other embodiments, the second member of a bio-orthogonal coupling pair can be a trans-cyclooctene (TCO).

The first member of a bio-orthogonal coupling pair can react with a second member of the bio-orthogonal coupling pair that is linked to a substrate, thereby producing polypeptides bound to the substrate. The substrate can be a bead or a solid surface. The substrate can be a magnetic bead. The substrate can be a bead contained within a chromatography column or a spin column. The substrate can be a porous matrix.

In certain embodiments, a linker links the first moiety and the second moiety. A linker is a moiety in a compound that connects one moiety to another. An “inert linker” is a moiety that covalently attaches, and optionally spaces, one moiety in a compound from another and which no substantial negative effect on the activity of the overall compound, e.g., in context of the present disclosure, the ability of the reactive groups, such as a tetrazine group, a succinimidyl group, or a dicarboxylic acid anhydride groups, such as maleic anhydride to react with their intended targets, and form and maintain a bond according to the methods described herein. Aside from serving to covalently-link two moieties, a linker may have a beneficial effect, such as in the physical separation of moieties to which it is attached, e.g., to optimize spacing to avoid steric effects. A linker also may serve some additional function, such as altering the hydrophobicity/hydrophilicity of the overall molecule, to provide an additional site, e.g., an amine protected by a protective group for linking additional moieties to the compound, or to rigidize the overall molecule. A linker is attached to the remainder of the compound by any suitable linkage moiety (“linkage”), e.g., by a carbon-carbon bond, an ester, a thioester, an amine, an ether, an amide, a carbonate, or a carbamate linkage to the additional moieties of the compound. The linker may be hydrocarbyl, that is including only carbons and hydrogens, or optionally comprising one or more hetero-atom, such as N, O, and/or S. In the context of the present disclosure, in one embodiment, one suitable linker is a divalent moiety comprising a PEG group-(O—CH2-CH2)n-, where n ranges from 2 to 100, e.g., from 2-15 (PEG₂₋₁₅), from 2-10 (PEG₂₋₁₀), or from 2-5 (PEG₂₋₅) such as 2, 3, 4 (PEG4), 5, 6, 7, 8, 9, 10, 11, 12, 13, 14, or 15. A PEG linker may comprise one or more methylene groups at either end in addition to a suitable linkages attaching the PEG group to the tetrazine and dicarboxylic anhydride moieties, e.g. maleic anhydride moieties.

In the methods disclosed herein, any starting sample can be used, such as a biological sample. The biological sample can be obtained from one biological source. The biological sample can be obtained from more than one biological source, such as in the case of pooled samples. The biological sample can be obtained from a patient. The biological sample can be obtained from a cell, tissue, or organ culture; a biopsy; pelleted cells from any biological fluid sample (i.e., urine, blood, saliva, mucus, cerebrospinal fluid, semen, aspirate); or a tissue culture for production of a recombinant protein, etc. The biological sample can be obtained by one of skill in the art using any means now known or later discovered including centrifugation, venipuncture, blood draw, excretion, swabbing, ejaculation, massage, biopsy, needle aspirate, lavage sample, scraping, surgical incision, laser capture microdissection, gradient separation, or other means known in the art.

As used herein, “single sample” refers to any biological sample from which DNA, RNA, and polypeptides are isolated/purified using the same method. For example, DNA, RNA, and polypeptides purified from a single sample means that the DNA, RNA, and polypeptides are all purified from the same biological sample during the same method disclosed herein. In contrast, in other conventional methods, DNA, RNA, and polypeptides are purified from the same biological sample by dividing the biological sample into multiple aliquots, and purifying DNA from one aliquot, RNA from another aliquot, and polypeptides from another aliquot using a different purification method for each aliquot.

In the disclosed methods, a cell lysate can be prepared, e.g., by use of any suitable method, as are broadly-known, e.g. by homogenization, optionally in the presence of any suitable salts, buffers, surfactants, emulsifiers, chaotropic agents, chelating agents, e.g., in urea, SDS or RIPA buffer. In certain embodiments, the mixed sample or cell lysate can be prepared in a solution comprising guanidinium thiocyanate. In certain embodiments, the mixed sample or cell lysate can be prepared by mechanical homogenization (e.g., mortar and pestle or passage through narrow-bore needles). In certain embodiments, the mixed sample or cell lysate can be prepared in a mildly-basic buffer or salt solution, e.g., to produce a lysate having a pH ranging from >7 to 10 (e.g., ranging from greater than 7 to 9 or 8 to 9.5). In certain other embodiments, the sample can be cell-free, and may not require lysing, such as in the case of analyzing a secretome, in the production of a recombinant protein that is secreted into the medium, or in the analysis of cell-free preparations (e.g. centrifuged supernatants) of biological fluids.

The method of purifying DNA, RNA, and polypeptides from a single sample disclosed herein can comprise the step of mixing a sample comprising DNA, RNA, and polypeptides with a compound disclosed herein (i.e., compound 1 or compound 2) thereby forming a mixed sample. When using compound 1 (e.g., mTet-PEGₙ-CDM), the mixed sample can comprise DNA, RNA, and pH dependent tagged polypeptides. When using compound 2 (e.g., mTet-PEGₙ-NHS), the mixed sample can comprise DNA, RNA, and permanently tagged polypeptides.

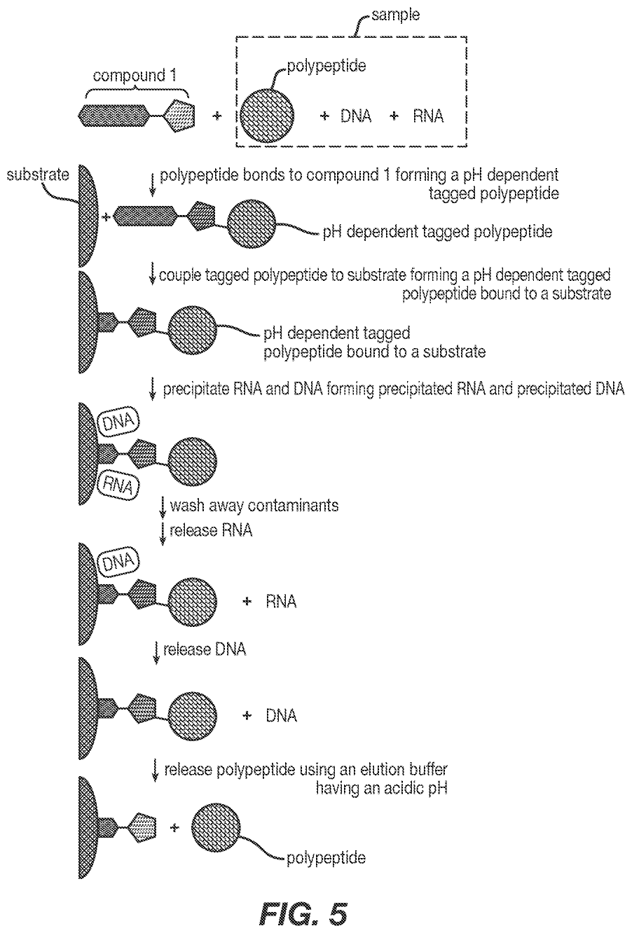

The method of purifying DNA, RNA, and polypeptides from a single sample disclosed herein can comprise the step of coupling pH dependent tagged polypeptides in a mixed sample to the second member of the bio-orthogonal coupling pair linked to a substrate, thereby forming pH dependent tagged polypeptides bound to a substrate, as illustrated in FIG. 5. In addition to the pH dependent tagged polypeptides, the DNA and RNA from the sample are also present, but are not shown in FIG. 5 until they are precipitated.

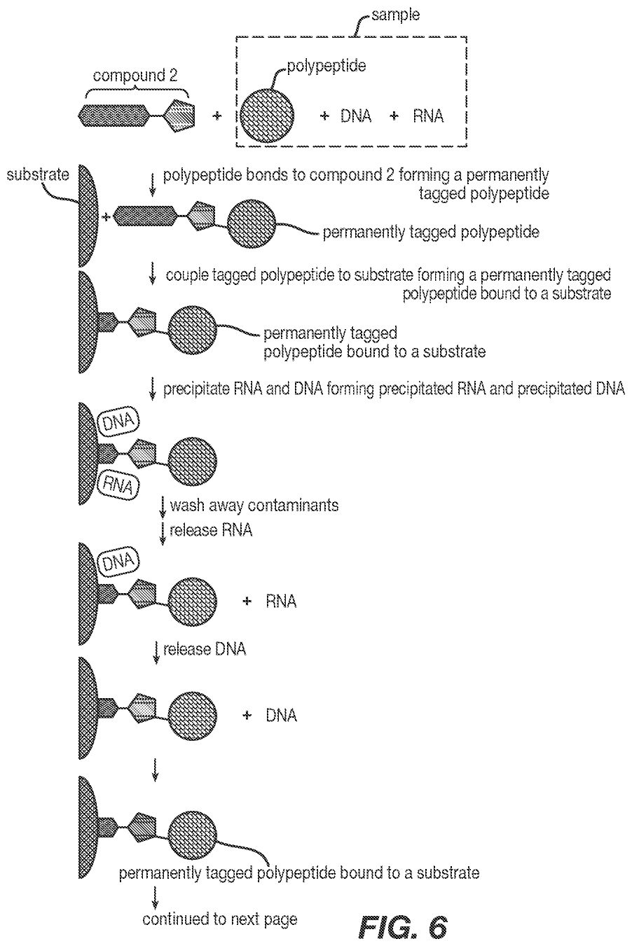

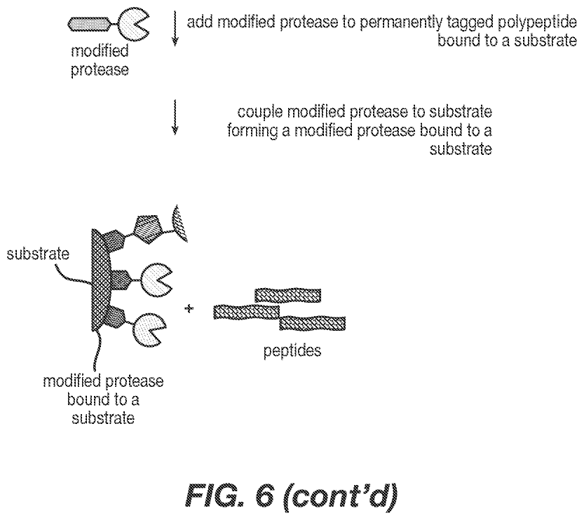

The method of purifying DNA, RNA, and polypeptides from a single sample disclosed herein can comprise the step of coupling permanently tagged polypeptides in a mixed sample to the second member of the bio-orthogonal coupling pair linked to a substrate, thereby forming permanently tagged polypeptides bound to a substrate, as illustrated in FIG. 6. In addition to the permanently tagged polypeptides, the DNA and RNA from the sample are present, but are not shown in FIG. 6 until they are precipitated.

As illustrated in FIG. 5 and FIG. 6, the method of purifying DNA, RNA, and polypeptides from a single sample disclosed herein can comprise the step of precipitating RNA and DNA in the mixed sample using a polar aprotic solvent thereby forming precipitated RNA and precipitated DNA. The RNA and DNA can be co-precipitated. The polar aprotic solvent can comprise acetonitrile or ethanol.

Without wishing to be bound by theory, the DNA and RNA are still in solution when the substrate (e.g. TCO beads) is added to the sample, allowing the tagged polypeptides to couple to the substrate (e.g. TCO beads). A polar aprotic solvent (e.g. acetonitrile, ethanol) is then added to precipitate the DNA and RNA. Prior to the addition of the polar aprotic solvent, the DNA and RNA do not interact with the substrate (e.g., TCO beads). After the addition of the polar aprotic solvent, the DNA and RNA precipitate forming aggregates that are approximately the same size as the beads. The DNA, RNA, and beads co-sediment together such that when separating the beads from free liquid, the DNA, RNA, and beads remain together in the column or spin filtration device, while the liquid moves through and out of the device.

In previous protein purification methods, including the methods disclosed in WO 2019/236988, high salt washes are used to wash away DNA and RNA, and other soluble contaminants. This step is followed by washes with acetonitrile to remove detergents, such as SDS. DNA, RNA, and detergents are removed during these methods because each interferes with mass spectrometry for peptide analysis. In previous protein purification methods, the DNA and RNA are treated as contaminants that should be removed. The goal of previous protein purification methods, including the methods disclosed in WO 2019/236988, was to only purify protein from a single sample and in the process of doing so—remove DNA and RNA, plus other contaminants.

There are at least three major differences between the protein purification methods and the multi-omics methods disclosed herein:
(1) Conventional protein purification methods only purify proteins from a single sample. The multi-omics method disclosed herein purifies DNA, RNA and polypeptides from a single sample.
(2) The lysis conditions and buffers are vastly different. For the conventional methods that only purify proteins, the samples are boiled in a 1-2% SDS buffer, which can denature all protein and damage the DNA and RNA. In the multi-omics methods disclosed herein, lysis conditions are less severe, which can prevent damage to the DNA and RNA. Instead of using SDS, guanidinium thiocyanate can be used in an example of the multi-omics methods disclosed herein. Instead of heating the sample, mechanical homogenization, such as mortar and pestle or passage through narrow-bore needles, can be used in an example of the multi-omics methods disclosed herein.
(3) The order and composition of the wash steps is also vastly different. The conventional methods that only purify proteins prioritize first ensuring the removal of DNA and RNA, and then removal of detergents. In contrast, the multi-omics methods disclosed herein maintain the DNA and RNA in an insoluble state while detergents and other contaminants are washed away. The DNA and RNA are not washed away. This is followed by controlled resolubilization of RNA, followed by resolubilization of the DNA. The controlled resolubilization of RNA followed by resolubilization of the DNA is achievable because RNA molecules are much smaller than DNA molecules, RNA is more soluble in low ionic strength, aqueous solutions compared to DNA, and DNA requires higher ionic strength solutions to be resolubilized.

As illustrated in FIG. 5, the method of purifying DNA, RNA, and polypeptides from a single sample disclosed herein can further comprise the step of washing the pH dependent tagged polypeptides that are bound to the substrate using any suitable wash solution that is not acidic (e.g., having a pH of 8 or greater). The pH of the wash solution for the pH dependent tagged polypeptides bound to a substrate can be selected to prevent premature hydrolysis of the amide bond attaching the pH dependent tagged polypeptides to the substrate. Suitable wash solutions include, without limitation: water, saline, PBS, Tris-EDTA, or other salt solutions or buffered salt solutions that do not hydrolyze the amide bond attaching the pH dependent tagged polypeptides to the substrate. For example, in certain embodiments, a polar aprotic solvent can be used to wash and remove any unbound materials (i.e. contaminants) from the pH dependent tagged polypeptides that are bound to the substrate.

As illustrated in FIG. 6, the method of purifying DNA, RNA, and polypeptides from a single sample disclosed herein can further comprise the step of washing the permanently tagged polypeptides that are bound to the substrate using any suitable wash solution. The pH of the wash solution for the permanently tagged polypeptides bound to a substrate can vary since the tag is permanent and there can be no premature hydrolysis of the amide bond attaching the permanently tagged polypeptides to the substrate. Suitable wash solutions include, without limitation: water, saline, PBS, Tris-EDTA, or other salt solutions or buffered salt solutions. In certain embodiments, a polar aprotic solvent can be used to wash and remove any unbound materials (i.e., contaminants) from the permanently tagged polypeptides that are bound to the substrate.

The substrates can be washed one or more times in an art-recognized manner for any selected substrate. Two or more wash steps, with the same or different wash solutions can be applied.

As illustrated in FIG. 5 and FIG. 6, the method of purifying DNA, RNA, and polypeptides from a single sample disclosed herein can comprise the step of resolubilizing and eluting precipitated RNA. The precipitated RNA can be resolubilized and eluted at room temperature using ultrapure water (available from Sigma Aldrich, Missouri, USA). The ultrapure water can be DNase free and RNase free.

As illustrated in FIG. 5 and FIG. 6, the method of purifying DNA, RNA, and polypeptides from a single sample disclosed herein can comprise the step of resolubilizing and eluting precipitated DNA. The precipitated DNA can be resolubilized and eluted at room temperature using a DNA elution buffer having a pH greater than 8, such as Tris-EDTA, or other salt solutions or buffered salt solutions that are able to elute the DNA, but do not hydrolyze the amide bond attaching the pH dependent tagged polypeptides to the substrate. Using a DNA elution buffer having a pH greater than 8 for those pH dependent tagged polypeptides bound to a substrate can prevent premature hydrolysis of the amide bond attaching the pH dependent tagged polypeptides to the substrate during the DNA elution step. In certain embodiments, a DNA elution buffer comprising 10 mM Tris, pH 8, 1 mM EDTA, and 10 mM NaCl can be used to elute the DNA without hydrolyzing the amide bond attaching the polypeptides to the substrate. The pH of the DNA elution buffer for the permanently tagged polypeptides bound to a substrate can vary since there can be no premature hydrolysis of the amide bond attaching the permanently tagged polypeptides to the substrate.

As illustrated in FIG. 5, the method of purifying DNA, RNA, and polypeptides from a single sample disclosed herein can comprise the step of eluting pH dependent tagged polypeptides bound to the substrate in an elution buffer having a pH more acidic than a pH of the mixed sample by reversing the pH dependent covalent bond between the polypeptides and the second moiety. By use of carboxylic anhydride coupling to amines of the polypeptides, once the amide bonds coupling the polypeptides to the substrate are cleaved, the primary amines of the polypeptides are restored. In certain embodiments, the elution buffer having a pH more acidic than a pH of the mixed sample can comprise a weak organic acid selected from the group consisting of formic acid, acetic acid, and citric acid. In certain other embodiments, the composition of the elution buffer can have a pH ranging from 2 to <7, 3 to <7 or from 2.5 to 6.

The method of purifying DNA, RNA, and polypeptides from a single sample disclosed herein can further comprise an optional step of mixing the polypeptides eluted in the elution buffer (e.g. polypeptides no longer bound to the substrate) with a modified protease thereby forming a mixed sample comprising peptides and the modified protease (this optional step is not illustrated in FIG. 5). The modified protease can be removed from the mixed sample by coupling the modified protease to a second member of a bio-orthogonal coupling pair, wherein the second member of the bio-orthogonal coupling pair is linked to the substrate. The peptides in the mixed sample can be eluted using any suitable elution buffer.

The modified protease can comprise a protease and a first member of a bio-orthogonal coupling pair attached to the protease. The first member can be configured to form a covalent bond with a second member of a bio-orthogonal coupling pair linked to the substrate, thereby producing protease bound to a substrate.

In certain embodiments, the modified protease can be a modified hydrolase and the protease can be a hydrolase. In certain other embodiments, the modified protease can be a modified serine hydrolase and the protease can be a serine hydrolase. In certain other embodiments, the modified protease can be a modified trypsin and the protease can be a trypsin.

Trypsin comprises a number of primary amines, e.g., Lys residues, and its N-terminus. The primary amine of lysine can be used to attach groups using any useful chemistry. Trypsin has 14 lysine residues, and only two arginine residues, the other target for autolysis. The modified trypsin is referred to herein as MT-trypsin. The presence of methyltetrazine on the MT-trypsin allows for the near complete removal of this protein from solution by coupling to beads containing TCO. Together, methyltetrazine and TCO form a bio-orthogonal coupling pair where these two moieties react to form a covalent linkage at very rapid rates. Other tetrazinyl moieties can be employed in this click chemistry pair, or other bio-orthogonal click chemistry pairs can be employed in the linking of trypsin to a substrate, such as a bead, for removal.

As illustrated in FIG. 6, the method of purifying DNA, RNA, and polypeptides from a single sample disclosed herein can comprise the step of adding a modified protease disclosed herein to permanently tagged polypeptides bound to the substrate, thereby forming peptides. The modified protease can be coupled to a second member of a bio-orthogonal coupling pair, wherein the second member of the bio-orthogonal coupling pair is linked to the substrate. The peptides can be eluted using any suitable elution buffer.

In certain embodiments, the method of purifying DNA, RNA, and polypeptides from a single sample disclosed herein resulted in yields of at least 75%, at least 80%, at least 85%, at least 90%, at least 95%, or at least 99% for polypeptides. In certain embodiments, the method of purifying DNA, RNA, and polypeptides from a single sample resulted in yields of at least 90%, at least 92.5%, at least 95%, at least 97.5%, at least 98%, at least 99%, at least 99.1%, at least 99.2%, at least 99.3%, at least 99.4%, at least 99.5%, at least 99.6%, at least 99.7%, or at least 99.8% clean reads for DNA. In certain embodiments, the method of purifying DNA, RNA, and polypeptides from a single sample resulted in yields of at least 90%, at least 92.5%, at least 95%, at least 97.5%, at least 98%, at least 99%, at least 99.1%, at least 99.2%, at least 99.3%, at least 99.4%, at least 99.5%, at least 99.6%, at least 99.7%, or at least 99.8% clean reads for RNA.

The quality of DNA and RNA purified using the method of purifying DNA, RNA, and polypeptides disclosed herein was analyzed (Novogene Corporation Inc., Sacramento, CA). In certain embodiments, the DNA had an error percent (error %) of 0.01, 0.02, 0.03, 0.04, or 0.05. In certain embodiments, the RNA had an error percent (error %) or error rate of 0.01, 0.02, 0.03, 0.04, or 0.05.

In another aspect of the present disclosure, a multi-omic kit is provided for purifying DNA, RNA, and polypeptides from a single sample. Disclosed herein is a multi-omic kit configured to perform any one of the methods of purifying DNA, RNA, and polypeptides from a sample disclosed herein. A multi-omic kit can comprise packaging and at least stated components of the kit. Packaging can be any suitable container, such as a box, sleeve, tube, carton, pouch, bag, etc., suitable for storage and/or delivery of the kit components. A “kit” may comprise one or more individual containers for the elements of the kit, though in one embodiment, all components of a kit are packaged together, or are packaged in a single container. For reagents or compositions, e.g. compounds, substrates, elution buffers, etc. described herein, the kit comprises one or more vessels containing stated reagent(s) or composition(s).

A multi-omic kit disclosed herein can comprise compound 1, a substrate linked to a second member of a bio-orthogonal coupling pair, and a polar aprotic solvent. The substrate can be used to isolate/purify DNA, RNA, polypeptides and optionally modified protease. The substrate can be in any suitable form, such as in the form of magnetic beads, beads or a porous matrix in a chromatography column, or a solid surface, such as a well of a multi-well plate. Additional vessels can comprise, for example, one or more of: a coupling solution disclosed herein, one or more wash solutions disclosed herein, and elution buffers disclosed herein, optionally in concentrated form, e.g., as a 2×, 5×, 10× or 25× concentrate. The coupling solution, DNA elution buffer, and RNA elution buffer can have a pH greater than 7, such as greater than 7 to 10, greater than 7 to 9, 8 to 9.5, or greater than 8 to 9.5. The elution buffer for eluting pH dependent tagged polypeptides from a substrate can have a pH less than 7, such as 2 to <7, 3 to <7, or 2.5 to 6.

Additional optional vessels can comprise a modified protease described herein and elution buffer for eluting peptides. The elution buffer for eluting peptides can have any pH between 7 and 9 (e.g., ammonium bicarbonate buffers). The pH is selected between 7 and 9 to be compatible with mass spectrometry.

A multi-omic kit disclosed herein can comprise compound 2, a substrate linked to a second member of a bio-orthogonal coupling pair, a polar aprotic solvent, and a modified protease described herein. The substrate can be used to isolate/purify DNA, RNA, polypeptides, and modified protease. The substrate can be in any suitable form, such as in the form of magnetic beads, beads or a porous matrix in a chromatography column, or a solid surface, such as a well of a multi-well plate. Additional vessels can comprise, for example, one or more of: a coupling solution disclosed herein, one or more wash solutions disclosed herein, and elution buffers disclosed herein, optionally in concentrated form, e.g., as a 2×, 5×, 10× or 25× concentrate. The pH of the coupling solution, DNA elution buffer, and RNA elution buffer does not have to be less than 7 for the permanently tagged polypeptides bound to a substrate since the tag is permanent and there can be no premature hydrolysis of the amide bond attaching the permanently tagged polypeptides to the substrate. Instead, these solutions and buffers can have any pH between 3 and 9. The elution buffer for eluting peptides can have any pH between 7 and 9 (e.g., ammonium bicarbonate buffers). The pH is selected between 7 and 9 to be compatible with mass spectrometry.

The vessels of the kit can be a compartment in a cartridge for use in an automated, or semi-automated device or system for purifying DNA, RNA, and polypeptide from a single sample.

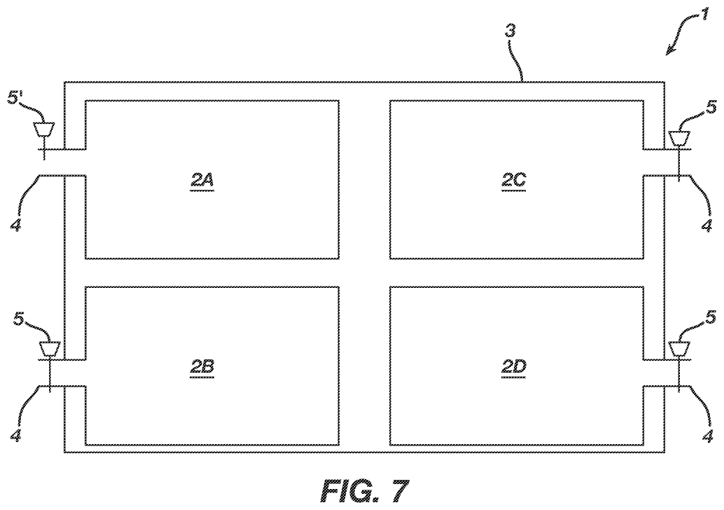

FIG. 7 depicts schematically a cartridge 1 comprising a housing 3, four compartments 2A, 2B, 2C, 2D, outlets 4, closed valves 5 and open valve 5′. The housing 5 can have any useful configuration and is adapted to insert into an automated device or system for controlling delivery of compositions contained within compartments 2A, 2B, 2C, 2D. Each compartment may contain a different reagent or composition, or the same reagent or composition. Valves 5 and 5′ may be controlled by any suitable mechanical or electromechanical mechanism, such as by solenoids and may be placed at any point in or external to the cartridge 1, for example, the outlets may fluidly couple with the valves, which are part of the device into which the cartridge 1 inserts. The cartridge 1 depicted in FIG. 7 is merely exemplary and may comprise any number of compartments, any shape, any fluid path, and any fluid control mechanism. A person of ordinary skill in the engineering arts can configure a suitable cartridge for use in any device or system, such as an automated system. Control of the cartridge and/or reagents or compositions removed from the cartridge, may be automated, e.g., controlled by a computer-implemented process.

The following numbered clauses are directed to various non-limiting embodiments of inventions according to the present disclosure:

Clause 1. A method of purifying DNA, RNA, and polypeptides from a sample, the method comprising:
a) mixing a sample comprising DNA, RNA, and polypeptides with a compound, thereby forming a mixed sample, wherein the compound comprises
   a first moiety comprising a first member of a bio-orthogonal coupling pair, wherein the first member is configured to form a covalent bond with a second member of a bio-orthogonal coupling pair;
   a second moiety configured to form a pH dependent covalent bond with a polypeptide; and
   a linker linking the first moiety and the second moiety;
b) coupling polypeptides in the mixed sample to the second member of the bio-orthogonal coupling pair, wherein the second member of the bio-orthogonal coupling pair is linked to a substrate, thereby forming polypeptides bound to the substrate;
c) precipitating RNA and DNA in the mixed sample using a polar aprotic solvent, thereby forming precipitated RNA and precipitated DNA;
d) resolubilizing and eluting the precipitated RNA;
e) resolubilizing and eluting the precipitated DNA; and
f) eluting the polypeptides bound to the substrate in an elution buffer having a pH more acidic than a pH of the mixed sample by reversing the pH dependent covalent bond between the polypeptides and the second moiety.

Clause 2. A method of purifying DNA, RNA, and polypeptides from a sample, the method comprising:
a) coupling polypeptides in a mixed sample to a second member of a bio-orthogonal coupling pair, wherein the second member of the bio-orthogonal coupling pair is linked to a substrate, thereby forming polypeptides bound to the substrate, and wherein the mixed sample comprises DNA, RNA, polypeptides, and a compound comprising
   a first moiety comprising a first member of a bio-orthogonal coupling pair, wherein the first member is configured to form a covalent bond with a second member of a bio-orthogonal coupling pair;
   a second moiety configured to form a pH dependent covalent bond with a polypeptide; and
   a linker linking the first moiety and the second moiety;
b) precipitating RNA and DNA in the mixed sample using a polar aprotic solvent, thereby forming precipitated RNA and precipitated DNA;
c) resolubilizing and eluting the precipitated RNA;
d) resolubilizing and eluting the precipitated DNA; and
e) eluting the polypeptides bound to the substrate in an elution buffer having a pH more acidic than a pH of the mixed sample by reversing the pH dependent covalent bond between the polypeptides and the second moiety.

Clause 3. The method of any one of clauses 1 or 2, further comprising:
mixing the polypeptides eluted in the elution buffer with a modified protease, thereby forming a mixed sample comprising peptides and the modified protease, wherein the modified protease comprises
   a protease; and
   a first member of a bio-orthogonal coupling pair attached to the protease, wherein the first member is configured to form a covalent bond with a second member of a bio-orthogonal coupling pair;
coupling the modified protease in the mixed sample to a second member of a bio-orthogonal coupling pair, wherein the second member of the bio-orthogonal coupling pair is linked to the substrate; and eluting the peptides.

Clause 4. The method of clause 3, wherein the modified protease is a modified hydrolase and the protease is a hydrolase.

Clause 5. The method of any one of clauses 3 or 4, wherein the modified protease is a modified serine hydrolase and the protease is a serine hydrolase.

Clause 6. The method of any one of clauses 3-5, wherein the modified protease is a modified trypsin and the protease is a trypsin.

Clause 7. The method of any one of clauses 1-6, wherein the second moiety is a dicarboxylic acid anhydride moiety.

Clause 8. The method of clause 7, wherein the dicarboxylic acid anhydride moiety is a maleic anhydride moiety.

Clause 9. The method of clause 7, wherein the dicarboxylic acid anhydride moiety is a 2-(2′-carboxyethyl) maleic anhydride moiety.

Clause 10. The method of any one of clauses 1-9, wherein the first member of the bio-orthogonal coupling pair is an electron-poor diene, an electron-rich dienophile, or a strained cycloalkene.

Clause 11. The method of any one of clauses 1-10, wherein the first member of a bio-orthogonal coupling pair is a tetrazine moiety selected from the group consisting of a 1,2,4,5-tetrazine moiety and a 4-(1,2,4,5-tetrazinyl)phenyl moiety.

Clause 12. The method of any one of clauses 1-11, wherein the linker is an inert linker.

Clause 13. The method of any one of clauses 1-12, wherein the substrate is a bead or a solid surface.

Clause 14. The method of any one of clauses 1-13, wherein the substrate is a magnetic bead.

Clause 15. The method of any one of clauses 1-14, wherein the substrate is a bead contained within a chromatography column or a spin column.

Clause 16. The method of any one of clauses 1-12, wherein the substrate is a porous matrix.

Clause 17. The method of any one of clauses 1-16, wherein the sample comprising DNA, RNA, and polypeptides is a cell or tissue lysate having a pH greater than 7.

Clause 18. The method of clause 17, wherein the cell or tissue lysate is prepared in a solution comprising guanidinium thiocyanate.

Clause 19. The method of any one of clauses 17 or 18, wherein the cell or tissue lysate is prepared by mechanical homogenization.

Clause 20. The method of any one of clauses 1-19, further comprising washing the polypeptides bound to the substrate using a polar aprotic solvent to remove any unbound materials from the polypeptides bound to the substrate.

Clause 21. The method of any one of clauses 1-20, wherein the polar aprotic solvent comprises acetonitrile or ethanol.

Clause 22. The method of any one of clauses 1-21, wherein the resolubilizing and eluting the precipitated RNA is achieved using an elution buffer comprising ultrapure water free of DNase and free of RNase.

Clause 23. The method of any one of clauses 1-22, wherein the resolubilizing and eluting precipitated DNA is achieved using an elution buffer having a pH greater than 7.

Clause 24. The method of any one of clauses 17-23, wherein the pH greater than 7 is from greater than 7 to 10, greater than 7 to 9, or 8 to 9.5.

Clause 25. The method of any one of clauses 1-24, wherein the elution buffer having a pH more acidic than a pH of the mixed sample comprises a weak organic acid selected from the group consisting of formic acid, acetic acid, and citric acid.

Clause 26. The method of any one of clauses 1-25, wherein the pH more acidic than a pH of the mixed sample is from 2 to less than 7, 3 to less than 7, or from 2.5 to 6.

Clause 27. A method of purifying DNA, RNA, and polypeptides from a sample, the method comprising:
a) mixing a sample comprising DNA, RNA, and polypeptides with a compound, thereby forming a mixed sample, wherein the compound comprises
   a first moiety comprising a first member of a bio-orthogonal coupling pair;
   a second moiety comprising dicarboxylic acid anhydride; and
   a linker linking the first moiety and the second moiety;
b) coupling polypeptides in the mixed sample to a second member of a bio-orthogonal coupling pair, wherein the second member of the bio-orthogonal coupling pair is linked to a substrate, thereby forming polypeptides bound to the substrate;
c) precipitating RNA and DNA in the mixed sample using a polar aprotic solvent, thereby forming precipitated RNA and precipitated DNA;
d) resolubilizing and eluting the precipitated RNA;
e) resolubilizing and eluting the precipitated DNA; and
f) eluting the polypeptides bound to the substrate in an elution buffer having a pH more acidic than a pH of the mixed sample by reversing a pH dependent covalent bond between the polypeptides and the second moiety.

Clause 28. A method of purifying DNA, RNA, and polypeptides from a sample, the method comprising:
a) mixing a sample comprising DNA, RNA, and polypeptides with a compound, thereby forming a mixed sample, wherein the compound comprises
   a first moiety comprising a first member of a bio-orthogonal coupling pair, wherein the first member is configured to form a covalent bond with a second member of a bio-orthogonal coupling pair;
   a second moiety configured to form a covalent bond with a polypeptide; and
   a linker linking the first moiety and the second moiety;
b) coupling polypeptides in the mixed sample to the second member of the bio-orthogonal coupling pair, wherein the second member of the bio-orthogonal coupling pair is linked to a substrate, thereby forming polypeptides bound to the substrate;
c) precipitating RNA and DNA in the mixed sample using a polar aprotic solvent, thereby forming precipitated RNA and precipitated DNA;
d) resolubilizing and eluting the precipitated RNA;
e) resolubilizing and eluting the precipitated DNA; and
f) adding a modified protease to the polypeptides bound to the substrate, thereby forming peptides,
   wherein the modified protease comprises
      a protease; and
      a first member of a bio-orthogonal coupling pair attached to the protease, wherein the first member is configured to form a covalent bond with a second member of a bio-orthogonal coupling pair;
g) coupling the modified protease to a second member of a bio-orthogonal coupling pair, wherein the second member of the bio-orthogonal coupling pair is linked to the substrate; and
h) eluting the peptides.

Clause 29. A method of purifying DNA, RNA, and polypeptides from a sample, the method comprising:
a) coupling polypeptides in a mixed sample to a second member of a bio-orthogonal coupling pair, wherein the second member of the bio-orthogonal coupling pair is linked to a substrate, thereby forming polypeptides bound to the substrate, wherein the mixed sample comprises DNA, RNA, polypeptides, and a compound comprising
   a first moiety comprising a first member of a bio-orthogonal coupling pair, wherein the first member is configured to form a covalent bond with a second member of a bio-orthogonal coupling pair;
   a second moiety configured to form a covalent bond with a polypeptide; and
   a linker linking the first moiety and the second moiety;
b) precipitating RNA and DNA in the mixed sample using a polar aprotic solvent, thereby forming precipitated RNA and precipitated DNA;
c) resolubilizing and eluting the precipitated RNA;
d) resolubilizing and eluting the precipitated DNA; and
e) adding a modified protease to the polypeptides bound to the substrate, thereby forming peptides,
   wherein the modified protease comprises
      a protease; and
      a first member of a bio-orthogonal coupling pair attached to the protease, wherein the first member is configured to form a covalent bond with a second member of a bio-orthogonal coupling pair;
f) coupling the modified protease to a second member of a bio-orthogonal coupling pair, wherein the second member of the bio-orthogonal coupling pair is linked to the substrate; and
g) eluting the peptides.

Clause 30. The method of any one of clauses 28 or 29, wherein the modified protease is a modified hydrolase and the protease is a hydrolase.

Clause 31. The method of any one of clauses 28-30, wherein the modified protease is a modified serine hydrolase and the protease is a serine hydrolase.

Clause 32. The method of any one of clauses 28-31, wherein the modified protease is a modified trypsin and the protease is a trypsin.

Clause 33. The method of any one of clauses 28-32, wherein the second moiety is N-hydroxysuccinimide.

Clause 34. The method of any one of clauses 28-33, wherein the first member of the bio-orthogonal coupling pair is an electron-poor diene, an electron-rich dienophile, or a strained cycloalkene.

Clause 35. The method of any one of clauses 28-34, wherein the first member of a bio-orthogonal coupling pair is a tetrazine moiety selected from the group consisting of a 1,2,4,5-tetrazine moiety and a 4-(1,2,4,5-tetrazinyl)phenyl moiety.

Clause 36. The method of any one of clauses 28-35, wherein the linker is an inert linker.

Clause 37. The method of any one of clauses 28-36, wherein the substrate is a bead or a solid surface.

Clause 38. The method of any one of clauses 28-37, wherein the substrate is a magnetic bead.

Clause 39. The method of any one of clauses 28-38, wherein the substrate is a bead contained within a chromatography column or a spin column.

Clause 40. The method of any one of clauses 28-36, wherein the substrate is a porous matrix.

Clause 41. The method of any one of clauses 28-40, wherein the sample comprising DNA, RNA, and polypeptides is a cell or tissue lysate.

Clause 42. The method of clause 41, wherein the cell or tissue lysate is prepared in a solution comprising guanidinium thiocyanate.

Clause 43. The method of any one of clauses 41 or 42, wherein the cell or tissue lysate is prepared by mechanical homogenization.

Clause 44. The method of any one of clauses 28-43, further comprising washing the polypeptides bound to the substrate using a polar aprotic solvent to remove any unbound materials from the polypeptides bound to the substrate.

Clause 45. The method of any one of clauses 28-44, wherein the polar aprotic solvent comprises acetonitrile or ethanol.

Clause 46. The method of any one of clauses 28-45 wherein the resolubilizing and eluting the precipitated RNA is achieved using an elution buffer comprising ultrapure water free of DNase and free of RNase.

Clause 47. The method of any one of clauses 28-46, wherein the resolubilizing and eluting the precipitated DNA is achieved using an elution buffer having a pH greater than 7.

Clause 48. The method of clause 47, wherein the pH greater than 7 ranges from greater than 7 to 10, greater than 7 to 9, or 8 to 9.5.

Clause 49. A method of purifying DNA, RNA, and polypeptides from a sample, the method comprising:
a) mixing a sample comprising DNA, RNA, and polypeptides with a compound, thereby forming a mixed sample, wherein the compound comprises
   a first moiety comprising a first member of a bio-orthogonal coupling pair;
   a second moiety comprising N-hydroxysuccinimide; and
   a linker linking the first moiety and the second moiety;
b) coupling polypeptides in the mixed sample to a second member of a bio-orthogonal coupling pair, wherein the second member of the bio-orthogonal coupling pair is linked to a substrate, thereby forming polypeptides bound to the substrate;
c) precipitating RNA and DNA in the mixed sample using a polar aprotic solvent, thereby forming precipitated RNA and precipitated DNA;
d) resolubilizing and eluting the precipitated RNA;
e) resolubilizing and eluting the precipitated DNA; and
f) adding a modified protease to the polypeptides bound to the substrate, thereby forming peptides,
   wherein the modified protease comprises
      a protease; and
      a first member of a bio-orthogonal coupling pair attached to the protease, wherein the first member is configured to form a covalent bond with a second member of a bio-orthogonal coupling pair;
g) coupling the modified protease to a second member of a bio-orthogonal coupling pair, wherein the second member of the bio-orthogonal coupling pair is linked to the substrate; and
h) eluting the peptides.

Clause 50. A method of purifying DNA, RNA, and polypeptides from a sample, the method comprising:
a) coupling polypeptides in a mixed sample to a second member of a bio-orthogonal coupling pair, wherein the second member of the bio-orthogonal coupling pair is linked to a substrate, thereby forming polypeptides bound to the substrate, wherein the mixed sample comprises DNA, RNA, polypeptides, and a compound comprising
   a first moiety comprising a first member of a bio-orthogonal coupling pair;
   a second moiety comprising N-hydroxysuccinimide; and
   a linker linking the first moiety and the second moiety;
b) precipitating RNA and DNA in the mixed sample using a polar aprotic solvent, thereby forming precipitated RNA and precipitated DNA;
c) resolubilizing and eluting the precipitated RNA;
d) resolubilizing and eluting the precipitated DNA; and
e) adding a modified protease to the polypeptides bound to the substrate, thereby forming peptides, wherein the modified protease comprises
   a protease; and
   a first member of a bio-orthogonal coupling pair attached to the protease, wherein the first member is configured to form a covalent bond with a second member of a bio-orthogonal coupling pair;
f) coupling the modified protease to a second member of a bio-orthogonal coupling pair, wherein the second member of the bio-orthogonal coupling pair is linked to the substrate; and
g) eluting the peptides.

Clause 51. A multi-omic kit configured to perform the method of any one of clauses 1-50.

Clause 52. A multi-omic isolation kit comprising
a compound comprising
   a first moiety comprising a first member of a bio-orthogonal coupling pair, wherein the first moiety is configured to form a covalent bond with a second member of a bio-orthogonal coupling pair;
   a second moiety configured to form a pH dependent covalent bond with a polypeptide; and
   a linker linking the first moiety and the second moiety;
a substrate linked to a second member of a bio-orthogonal coupling pair, wherein the substrate is used to isolate DNA, RNA, and polypeptides; and
a polar aprotic solvent.

Clause 53. The multi-omic isolation kit of clause 52, further comprising
a modified protease comprising
   a protease; and
   a first member of a bio-orthogonal coupling pair attached to the protease, wherein the first member is configured to form a covalent bond with a second member of a bio-orthogonal coupling pair.

Clause 54. The multi-omic isolation kit of any one of clauses 52 or 53, wherein the modified protease is a modified hydrolase and the protease is a hydrolase.

Clause 55. The multi-omic isolation kit of any one of clauses 52-54, wherein the modified protease is a modified serine hydrolase and the protease is a serine hydrolase.

Clause 56. The multi-omic isolation kit of any one of clauses 52-55, wherein the modified protease is a modified trypsin and the protease is a trypsin.

Clause 57. The multi-omic isolation kit of any one of clauses 52-56, wherein the second moiety is a dicarboxylic acid anhydride moiety.

Clause 58. The multi-omic isolation kit of clause 57, wherein the dicarboxylic acid anhydride moiety is a maleic anhydride moiety.

Clause 59. The multi-omic isolation kit of clause 57, wherein the dicarboxylic acid anhydride moiety is a 2-(2′-carboxyethyl) maleic anhydride moiety.

Clause 60. The multi-omic isolation kit of any one of clauses 52-59, wherein the first member of a bio-orthogonal coupling pair is an electron-poor diene, an electron-rich dienophile, or a strained cycloalkene.

Clause 61. The multi-omic isolation kit of any one of clauses 52-60, wherein the first member of a bio-orthogonal coupling pair is a tetrazine moiety selected from the group consisting of a 1,2,4,5-tetrazine moiety or a 4-(1,2,4,5-tetrazinyl)phenyl moiety.

Clause 62. The multi-omic isolation kit of any one of clauses 52-61, wherein the substrate is a bead, a solid surface, or a porous matrix.

Clause 63. The multi-omic isolation kit of any one of clauses 52-62, wherein the substrate is a bead, a solid surface, or a porous matrix contained in a chromatography column or a spin column.

Clause 64. The multi-omic isolation kit of any one of clauses 52-63, wherein the substrate is a magnetic bead.

Clause 65. The multi-omic isolation kit of any one of clauses 52-64, wherein the polar aprotic solvent comprises acetonitrile or ethanol.

Clause 66. A multi-omic isolation kit comprising
a compound comprising
   a first moiety comprising a first member of a bio-orthogonal coupling pair, wherein the first moiety is configured to form a covalent bond with a second member of a bio-orthogonal coupling pair;
   a second moiety configured to form a covalent bond with a polypeptide; and
   a linker linking the first moiety and the second moiety;
a substrate linked to a second member of a bio-orthogonal coupling pair, wherein the substrate is used to isolate DNA, RNA, polypeptides, and modified protease;
a polar aprotic solvent; and
a modified protease comprising
   a protease; and
   a first member of a bio-orthogonal coupling pair attached to the protease, wherein the first member is configured to form a covalent bond with a second member of a bio-orthogonal coupling pair.

Clause 67. The multi-omic isolation kit of clause 66, wherein the modified protease is a modified hydrolase and the protease is a hydrolase.

Clause 68. The multi-omic isolation kit of any one of clauses 66 or 67, wherein the modified protease is a modified serine hydrolase and the protease is a serine hydrolase.

Clause 69. The multi-omic isolation kit of any one of clauses 66-68, wherein the modified protease is a modified trypsin and the protease is a trypsin.

Clause 70. The multi-omic isolation kit of any one of clauses 66-69, wherein the second moiety is N-hydroxysuccinimide.

Clause 71. The multi-omic isolation kit of any one of clauses 66-70, wherein the first member of a bio-orthogonal coupling pair is an electron-poor diene, an electron-rich dienophile, or a strained cycloalkene.

Clause 72. The multi-omic isolation kit of any one of clauses 66-71, wherein the first member of a bio-orthogonal coupling pair is a tetrazine moiety selected from the group consisting of a 1,2,4,5-tetrazine moiety or a 4-(1,2,4,5-tetrazinyl)phenyl moiety.

Clause 73. The multi-omic isolation kit of any one of clauses 66-72, wherein the substrate is a bead, a solid surface, or a porous matrix.

Clause 74. The multi-omic isolation kit of any one of clauses 66-73, wherein the substrate is a bead, a solid surface, or a porous matrix contained in a chromatography column or a spin column.

Clause 75. The multi-omic isolation kit of any one of clauses 66-74, wherein the substrate is a magnetic bead.

Clause 76. The multi-omic isolation kit of any one of clauses 66-75, wherein the polar aprotic solvent comprises acetonitrile or ethanol.

### Definitions

In addition to the definitions previously set forth herein, the following definitions are relevant to the present disclosure.

As used herein, “tag”, “tagged”, or “tagging” refers to coupling compound 1 or compound 2 to amines of a polypeptide, thereby bonding the compound to the polypeptide. For example, carboxylic anhydride of compound 1 forms a pH dependent covalent bond with the amines of the polypeptide. For example, N-hydroxysuccinimide of compound 2 forms a permanent covalent bond with the amines of the polypeptide.

As used herein, “pH dependent-tagged polypeptide” refers to a polypeptide with a pH dependent covalent bond between amines of the polypeptide and compound 1. For example, a pH dependent-tagged polypeptide can have a pH dependent covalent bond between amines of the polypeptide and carboxylic anhydride of compound 1. The pH dependent covalent bond between amines of the polypeptide and carboxylic anhydride of compound 1 can be reversed (i.e., cleaved) by using an elution buffer with a pH less than 7 (e.g., 2 to less than 7, 3 to less than 7, or from 2.5 to 6).

As used herein, “permanently-tagged polypeptide” refers to a polypeptide with a permanent covalent bond between amines of the polypeptide and compound 2. For example, a permanently-tagged polypeptide can have a permanent covalent bond between amines of the polypeptide and N-hydroxysuccinimide of compound 2. The permanent covalent bond between amines of the polypeptide and carboxylic anhydride of compound 2 is stable and cannot be reversed at a pH of 2 to 12, a temperature of less than 100° C., and an exposure time of less than 24 hours. Instead, the permanent covalent bond can only be broken by exposing the permanently tagged polypeptides to extreme conditions outside the pH, temperature and time ranges described above, such as for example, boiling the permanently-tagged polypeptides in 6N hydrochloric acid for 24 hours.

A “moiety” (pl. “moieties”) is a part of a chemical compound, and includes groups, such as functional groups. As such, a nucleobase moiety is a nucleobase that is modified by attachment to another compound moiety, such as a polymer monomer, e.g. the nucleic acid or nucleic acid analog monomers described herein, or a polymer, such as a nucleic acid or nucleic acid analog as described herein.

A “polypeptide” includes proteins and oligopeptides as a class, and generally refers to a polypeptide comprising two or more amino acid residues, though typically referring to longer amino acid chains.

As used herein, “at least one of” a list of elements means one of the elements or any combination of two or more of the listed elements. As an example “at least one of A, B, and C” means A only; B only; C only; A and B; A and C; B and C; or A, B, and C.

Various features and characteristics are described in this specification to provide an understanding of the composition, structure, production, function, and/or operation of the invention, which includes the disclosed compositions, kits, and methods. It is understood that the various features and characteristics of the invention described in this specification can be combined in any suitable manner, regardless of whether such features and characteristics are expressly described in combination in this specification. The Inventors and the Applicant expressly intend such combinations of features and characteristics to be included within the scope of the invention described in this specification. As such, the claims can be amended to recite, in any combination, any features and characteristics expressly or inherently described in, or otherwise expressly or inherently supported by, this specification. Furthermore, the Applicant reserves the right to amend the claims to affirmatively disclaim features and characteristics that may be present in the prior art, even if those features and characteristics are not expressly described in this specification. Therefore, any such amendments will not add new matter to the specification or claims and will comply with the written description, sufficiency of description, and added matter requirements.

Any numerical range recited in this specification describes all sub-ranges of the same numerical precision (i.e., having the same number of specified digits) subsumed within the recited range. For example, a recited range of “1.0 to 10.0” describes all sub-ranges between (and including) the recited minimum value of 1.0 and the recited maximum value of 10.0, such as, for example, “2.4 to 7.6,” even if the range of “2.4 to 7.6” is not expressly recited in the text of the specification. Accordingly, the Applicant reserves the right to amend this specification, including the claims, to expressly recite any sub-range of the same numerical precision subsumed within the ranges expressly recited in this specification. All such ranges are inherently described in this specification such that amending to expressly recite any such sub-ranges will comply with the written description, sufficiency of description, and added matter requirements.

Also, unless expressly specified or otherwise required by context, all numerical parameters described in this specification (such as those expressing values, ranges, amounts, percentages, and the like) may be read as if prefaced by the word “about,” even if the word “about” does not expressly appear before a number. Additionally, numerical parameters described in this specification should be construed in light of the number of reported significant digits, numerical precision, and by applying ordinary rounding techniques. It is also understood that numerical parameters described in this specification will necessarily possess the inherent variability characteristic of the underlying measurement techniques used to determine the numerical value of the parameters.

Notwithstanding that numerical ranges and parameters setting forth the broad scope of the invention are approximations, numerical values set forth in the specific examples are reported precisely as possible. Any numerical value, however, inherently contains certain errors necessarily resulting from the standard variation found in its respective testing measurements.

Reference throughout the specification to “certain embodiments,” “certain other embodiments,” “one embodiment,” “an embodiment,” or the like means that a particular feature, structure, step, or characteristic described in connection with the example is included in at least one embodiment. Thus, appearances of the phrases “certain embodiments,” “certain other embodiments,” “one embodiment,” “an embodiment,” or the like, in places throughout the specification are not necessarily all referring to the same embodiment. Furthermore, the particular described features, structures, steps, or characteristics may be combined in any suitable manner in one or more embodiments. Thus, the particular features, structures, steps, or characteristics illustrated or described in connection with one embodiment may be combined, in whole or in part, with the features, structures, steps, or characteristics of one or more other embodiments without limitation. Such modifications and variations are intended to be included within the scope of the present embodiments.

Any patent, publication, or other document identified in this specification is incorporated by reference into this specification in its entirety unless otherwise indicated but only to the extent that the incorporated material does not conflict with existing descriptions, definitions, statements, illustrations, or other disclosure material expressly set forth in this specification. As such, and to the extent necessary, the express disclosure as set forth in this specification supersedes any conflicting material incorporated by reference. Any material, or portion thereof, that is incorporated by reference into this specification, but which conflicts with existing definitions, statements, or other disclosure material set forth herein, is only incorporated to the extent that no conflict arises between that incorporated material and the existing disclosure material. Applicant reserves the right to amend this specification to expressly recite any subject matter, or portion thereof, incorporated by reference. The amendment of this specification to add such incorporated subject matter will comply with the written description, sufficiency of description, and added matter requirements.

It is understood that the inventions described in this specification are not limited to the examples summarized in the Summary or Detailed Description. Various other aspects are described and exemplified herein.

### EXAMPLES

The present disclosure will be more fully understood by reference to the following examples, which provide illustrative, non-limiting aspects of the present disclosure.

### Example 1—Coupling pH Dependent Tagged Polypeptides in a Cell Lysate to a Substrate

A cell or tissue sample was centrifuged in a benchtop centrifuge to form a cell pellet. Lysis buffer (100 mM NaCl, 100 mM HEPES pH 8, 4 M guanidine thiocyanate) was added to the cell pellet and mixed, thereby forming a cell lysate. The cell lysate was homogenized by passing it through a 21-gauge needle, 10 times. The protein concentration of the cell lysate was assayed using BCA Protein Assay (available from Thermo Fisher Scientific, Pennsylvania, USA).

The cell lysate including 100 μg protein was mixed with 2.1 μl of 30 mg/ml mTet-PEGₙ-CDM in 90% acetonitrile, 0.1% trifluoracetic acid (see FIG. 2) by pipetting the mixture gently and then incubating it for 30 minutes at 4° C. to form pH dependent tagged polypeptides in the cell lysate.

After 30 minutes, the mixed cell lysate (which includes DNA, RNA, and the pH dependent tagged polypeptides) was added directly to a capture resin positioned within a resin capture tube. The resin capture tube is a spin column tube with a fine slit in the tip of the centrifuge tube. The slit is so fine that TCO-beads and precipitated DNA and RNA are retained within the tube while liquid passes through the slit under centrifugal force. A second member of a bio-orthogonal coupling pair (e.g. TCO) was linked to the capture resin. The second member forms a covalent bond with the first member of a bio-orthogonal coupling pair of the pH dependent tagged polypeptides.

175 μl of 100% acetonitrile was added to the capture resin. The resin capture tube was then incubated at 4° C. with gentle rotation for 30 minutes, thereby forming pH dependent tagged polypeptides bound to the capture resin.

### Example 2—Precipitating DNA and RNA and Purifying the DNA and RNA

The resin capture tube in Example 1 was inserted into a waste collection tube and centrifuged briefly for 2-6 seconds in a benchtop centrifuge until all of the liquid had passed into the waste collection tube.

Next, a washing step was performed by adding 200 μl of a washing buffer (70% acetonitrile) to the resin capture tube positioned within the waste collection tube, vortexing it for 1 second, and centrifuging it briefly for 2-6 seconds in a benchtop centrifuge until all of the liquid had passed through into the waste collection tube. This washing step was repeated 2 more times.

30 μl of RNA elution buffer, which is DNase free and RNase free ultrapure water, was directly added to the capture resin in the resin capture tube. The resin capture tube was gently tapped to mix its contents and then incubated at 4° C. with gentle rotation for 5 minutes. The resin capture tube was inserted into a nucleic acid collection tube and centrifuged for 2-6 seconds in a benchtop centrifuge until all of the liquid had passed through into the nucleic acid collection tube. The liquid that passed into the nucleic acid collection tube included purified RNA. DNase was added to the nucleic acid collection tube to remove any contaminating DNA from the purified RNA. The purified RNA was stored at −80° C.

30 μl of DNA elution buffer (10 mM Tris pH 8, 1 mM EDTA, 10 mM NaCl) was then directly added to the capture resin in the resin capture tube. The resin capture tube was gently tapped to mix its contents and then incubated at 4° C. with gentle rotation for 5 minutes. The resin capture tube was inserted into a nucleic acid collection tube and centrifuged for 2-6 seconds in a benchtop centrifuge until all of the liquid had passed into the nucleic acid collection tube. The liquid that passed into the nucleic acid collection tube included purified DNA. RNase was added to the nucleic acid collection tube to remove any contaminating RNA from the purified DNA. The purified DNA was stored at −80° C.

A washing step was performed by adding 200 μl of wash buffer #1 (250 mM NaCl, 10% acetonitrile, 100 mM HEPES pH 8) to the resin capture tube positioned within a waste collection tube, vortexing it for 1 second, and centrifuging it briefly for 2-6 seconds in a benchtop centrifuge until all of the liquid had passed through into the waste collection tube. The liquid in the waste collection tube was discarded.

A second washing step was performed by adding 200 μl of wash buffer #2 (75% acetonitrile, 25% 100 mM HEPES pH 8) to the resin capture tube positioned within a waste collection tube, vortexing it for 1 second, and centrifuging it briefly for 2-6 seconds in a benchtop centrifuge until all of the liquid had passed into the waste collection tube. The liquid in the waste collection tube was discarded.

A final washing step was performed by adding 200 μl of ultrapure water to the resin capture tube positioned within a waste collection tube, vortexing it for 1 second, and centrifuging it briefly for 2-6 seconds in a benchtop centrifuge until all of the liquid had passed into the waste collection tube. The liquid in the waste collection tube was discarded. The final washing step was performed one additional time.

The eluted DNA and RNA were further analyzed by PCR to determine whether it was free of degradation. The DNA and RNA was further analyzed using 260/280 absorption and/or WGS to confirm its purity.

### Example 3—Removing pH Dependent Tagged Polypeptides from the Substrate

After the final washing step in Example 2, 50 μl of elution buffer (100 mM formic acid) was added to the capture resin positioned within the resin capture tube. The tube was incubated at room temperature with gentle rotation for 15 minutes to release the polypeptides from the capture resin.

### Example 4—Protein Digestion of pH Dependent Tagged Polypeptides Released from the Substrate

After the 15-minute incubation at room temperature in Example 3, the resin capture tube was inserted into a low protein binding collection tube and 25 μl of a modified trypsin was directly added to the capture resin. The resin capture tube was gently tapped to mix its contents and then incubated at 37° C. for 1 hour. After 1 hour, the resin capture tube positioned in the low protein binding collection tube was centrifuged for 2-6 seconds in a benchtop centrifuge until all of the liquid had passed into the low protein binding collection tube. The liquid that passed into the low protein binding collection tube included peptides.

50 μl of elution buffer elution (100 mM formic acid) was directly added to the capture resin in the resin capture tube. The resin capture tube was gently tapped to mix its contents and then incubated at room temperature with gentle rotation for 5 minutes. The resin capture tube was inserted back into the low protein binding collection tube and centrifuged for 5-10 seconds in a benchtop centrifuge until all of the liquid had passed into the low protein binding collection tube. This additional liquid that passed into the low protein binding collection tube also included peptides.

The peptides were further analyzed by Liquid Chromatography-Mass Spectrometry (LC-MS/MS) to determine the mass of the peptides and their purity. MS was also used to determine the amino acid sequence of the peptides to further characterize the proteins being studied or identified.

### Example 5—Coupling Permanently Tagged Polypeptides in a Cell Lysate to a Substrate

A cell or tissue sample was centrifuged in a benchtop centrifuge to form a cell pellet. Lysis buffer (100 mM NaCl, 100 mM HEPES pH 8, 4 M guanidine thiocyanate) was added to the cell pellet and mixed, thereby forming a cell lysate. The cell lysate was homogenized by passing it through a 21-gauge needle, 10 times. The protein concentration of the cell lysate was assayed using BCA Protein Assay (available from Thermo Fisher Scientific, Pennsylvania, USA).

The cell lysate including 100 μg protein was mixed with 2.1 μl of 30 mg/ml mTet-PEGₙ-NHS in dimethylformamide (see FIG. 4) by pipetting the mixture gently and then incubating it for 30 minutes at 4° C. to form permanently tagged polypeptides in the cell lysate.

After 30 minutes, the mixed cell lysate (which includes DNA, RNA, and permanently tagged polypeptides) was added directly to a capture resin positioned within a resin capture tube. A second member of a bio-orthogonal coupling pair (e.g. TCO) was linked to the capture resin. The second member forms a covalent bond with the first member of a bio-orthogonal coupling pair of the permanently tagged polypeptides.

175 μl of 100% acetonitrile was added to the capture resin. The resin capture tube was then incubated at 4° C. with gentle rotation for 30 minutes, thereby forming permanently tagged polypeptides bound to the capture resin.

### Example 6—Precipitating DNA and RNA and Purifying the DNA and RNA

The resin capture tube in Example 5 was inserted into a waste collection tube and centrifuged briefly for 2-6 seconds in a benchtop centrifuge until all of the liquid had passed into the waste collection tube.

Next, a washing step was performed by adding 200 μl of a washing buffer (70% acetonitrile) to the resin capture tube positioned within the waste collection tube, vortexing it for 1 second, and centrifuging it briefly for 2-6 seconds in a benchtop centrifuge until all of the liquid had passed through into the waste collection tube. This washing step was repeated 2 more times.

30 μl of RNA elution buffer, which is DNase free and RNase free ultrapure water, was directly added to the capture resin in the resin capture tube. The resin capture tube was gently tapped to mix its contents and then incubated at 4° C. with gentle rotation for 5 minutes. The resin capture tube was inserted into a nucleic acid collection tube and centrifuged for 2-6 seconds in a benchtop centrifuge until all of the liquid had passed through into the nucleic acid collection tube. The liquid that passed into the nucleic acid collection tube included purified RNA. DNase was added to the nucleic acid collection tube to remove any contaminating DNA from the purified RNA. The purified RNA was stored at −80° C.

30 μl of DNA elution buffer (10 mM Tris pH 8, 1 mM EDTA, 10 mM NaCl) was then directly added to the capture resin in the resin capture tube. The resin capture tube was gently tapped to mix its contents and then incubated at 4° C. with gentle rotation for 5 minutes. The resin capture tube was inserted into a nucleic acid collection tube and centrifuged for 2-6 seconds in a benchtop centrifuge until all of the liquid had passed into the nucleic acid collection tube. The liquid that passed into the nucleic acid collection tube included purified DNA. RNase was added to the nucleic acid collection tube to remove any contaminating RNA from the purified DNA. The purified DNA was stored at −80° C.

A washing step was performed by adding 200 μl of wash buffer #1 (250 mM NaCl, 10% acetonitrile, 100 mM HEPES pH 8) to the resin capture tube positioned within a waste collection tube, vortexing it for 1 second, and centrifuging it briefly for 2-6 seconds in a benchtop centrifuge until all of the liquid had passed through into the waste collection tube. The liquid in the waste collection tube was discarded.

A second washing step was performed by adding 200 μl of wash buffer #2 (75% acetonitrile, 25% 100 mM HEPES pH 8) to the resin capture tube positioned within a waste collection tube, vortexing it for 1 second, and centrifuging it briefly for 2-6 seconds in a benchtop centrifuge until all of the liquid had passed into the waste collection tube. The liquid in the waste collection tube was discarded.

A final washing step was performed by adding 200 μl of ultrapure water to the resin capture tube positioned within a waste collection tube, vortexing it for 1 second, and centrifuging it briefly for 2-6 seconds in a benchtop centrifuge until all of the liquid had passed into the waste collection tube. The liquid in the waste collection tube was discarded. The final washing step was performed one additional time.

The DNA and RNA were further analyzed by PCR to determine whether it was free of degradation. The DNA and RNA was further analyzed using 260/280 absorption and/or WGS to confirm its purity.

### Example 7—Protein Digestion of Permanently Tagged Polypeptides Bound to a Substrate

After the final washing step in Example 6, the resin capture tube was inserted into a low protein binding collection tube and 25 μl of a modified trypsin was directly added to the capture resin. The resin capture tube was gently tapped to mix its contents and then incubated at 37° C. for 1 hour. After 1 hour, the resin capture tube positioned in the low protein binding collection tube was centrifuged for 2-6 seconds in a benchtop centrifuge until all of the liquid had passed into the low protein binding collection tube. The liquid that passed into the low protein binding collection tube included peptides.

50 μl of elution buffer elution (100 mM formic acid) was directly added to the capture resin in the resin capture tube. The resin capture tube was gently tapped to mix its contents and then incubated at room temperature with gentle rotation for 5 minutes. The resin capture tube was inserted back into the low protein binding collection tube and centrifuged for 5-10 seconds in a benchtop centrifuge until all of the liquid had passed into the low protein binding collection tube. This additional liquid that passed into the low protein binding collection tube also included peptides.

The peptides were further analyzed by Liquid Chromatography-Mass Spectrometry (LC-MS/MS) to determine the mass of the peptides and their purity. MS was also used to determine the amino acid sequence of the peptides to further characterize the proteins being studied or identified.

### Example 8—Elution of Unmodified Proteins from Trans-Cyclooctene (TCO) Beads

Model proteins, such as Lysozyme, Carbonic Anhydrase (CA), Alcohol Dehydrogenase (ADH) and a yeast lysate were mixed with mTet-PEGₙ-CDM (see FIG. 2), thereby forming pH dependent tagged polypeptides. The pH dependent tagged polypeptides were added to TCO beads, thereby forming pH dependent tagged polypeptides bound to TCO beads, as described in Example 1. After extensive washing, the proteins were eluted from the TCO beads as described in Example 3 by reversal of the polypeptide-CDM linkage. This releases proteins in their original, unmodified state.

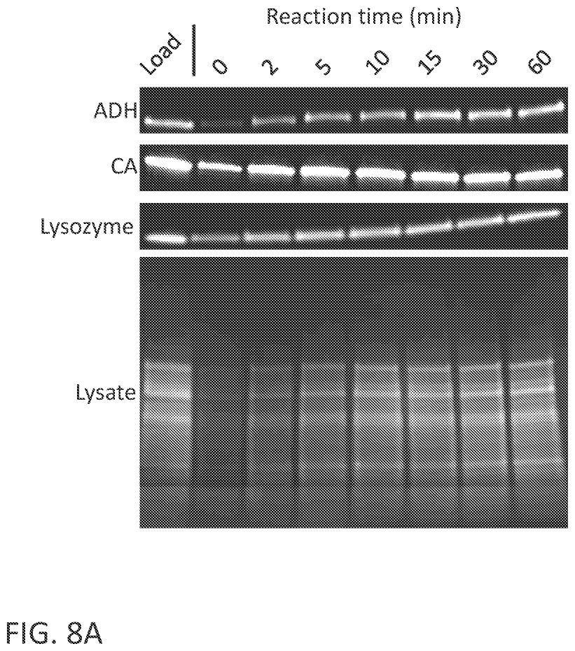

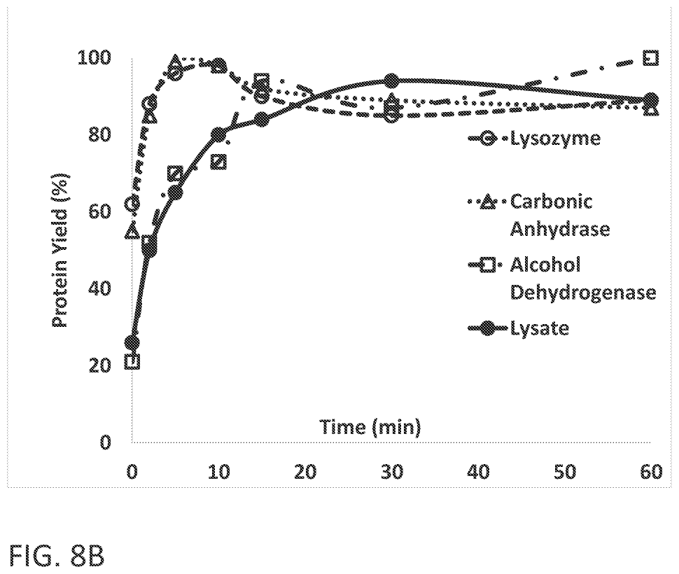

Each of the model proteins (Lysozyme, CA, and ADH) were recovered from mTet-PEGₙ-CDM tagged polypeptides coupled to TCO beads at 0, 2, 5, 10, 15, 30, and 60 minutes after lowering the pH to at or near 3. Proteins from the yeast lysate were also recovered from mTet-PEGₙ-CDM tagged polypeptides coupled to TCO beads at 0, 2, 5, 10, 15, 30, and 60 minutes after lowering the pH to at or near 3. See FIG. 8A. After 10 minutes, greater than 80% of the proteins were released from the TCO beads. After 30 minutes, greater than 90% of the proteins were released from the TCO beads. See FIG. 8B.

These experiments in FIGS. 8A and 8B demonstrate that mTet-PEG-CDM can be used as a universal protein tag that is capable of (1) capturing more than 85% of protein in a solution, (2) forming a sufficiently stable covalent bond with a substrate to extensively wash away non-binding contaminants, and (3) releasing the captured and cleaned protein with yields greater than 85%.

### Example 9—Mass Spectrometry Analysis of a Yeast Proteome

Three separate yeast cell lysate samples (A, B, and C) were prepared and mixed with mTet-PEGₙ-CDM, thereby forming pH dependent tagged polypeptides, as described in Example 1. The mixed yeast cell lysates (including the pH dependent tagged polypeptides) were then added to TCO beads, as described in Example 1. After extensive washing, the proteins were eluted from the TCO beads by reversal of the polypeptide-CDM linkage, as described in Example 3. The eluted proteins were then digested, as described in Example 4, thereby forming peptides.

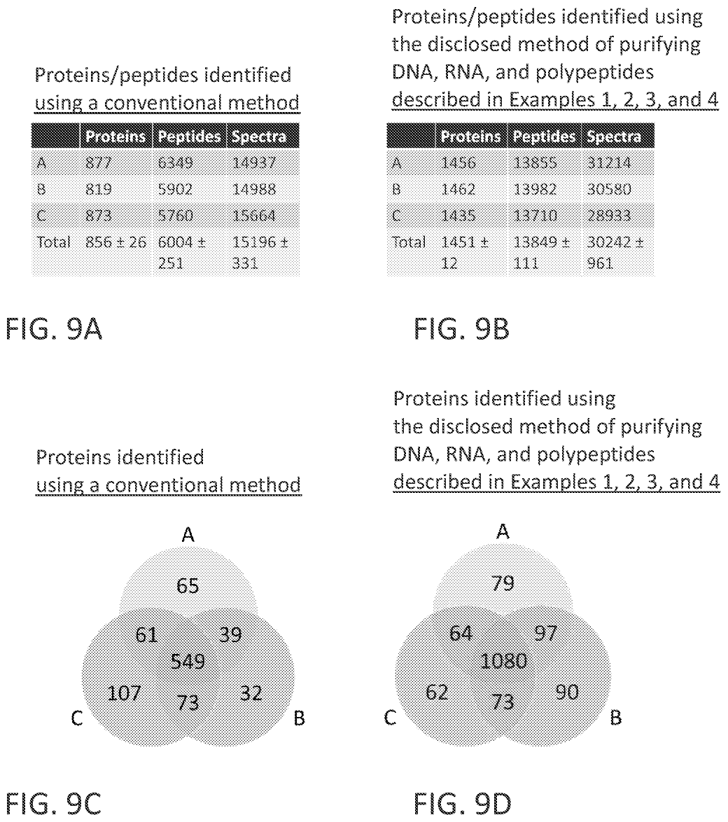

The peptides for each of sample A, B, and C were analyzed by Liquid Chromatography-Mass Spectrometry (LC-MS/MS) and compared to peptides of samples A, B, and C prepared by a conventional method (the conventional method being acetone precipitation of proteins). See FIGS. 9A, 9C and FIGS. 9B, 9D. In FIGS. 9C and 9D, overlap of the proteins identified in each cell lysate sample (A, B, C) is based on the p-value score.

These experiments further demonstrate that the multi-omic method of purifying DNA, RNA, and polypeptides disclosed herein is both high yield and highly repeatable.

### Example 10—Analysis of Purified Yeast Genomic DNA

An experiment was performed to assess the quality of DNA recovered from the method of purifying DNA, RNA, and polypeptides disclosed herein.

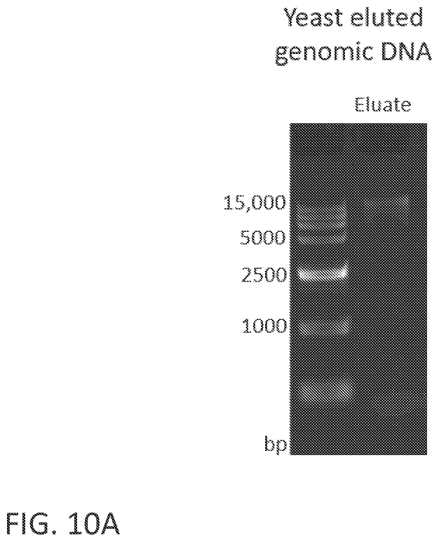

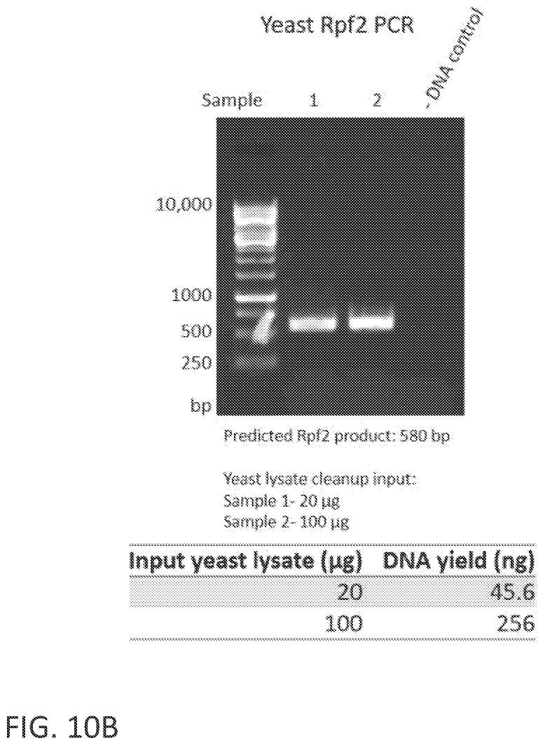

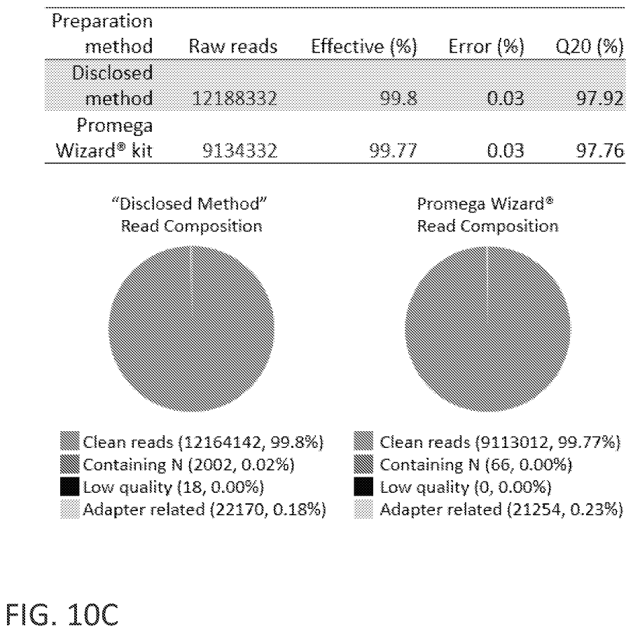

Yeast cell lysates were prepared as described in Example 1. Yeast cell lysates including either 20 μg or 100 μg of protein were used to prepare purified DNA, as described in Example 2. The eluted yeast genomic DNA shown in FIG. 10A demonstrates the purified yeast genomic DNA is free of degradation.

The purified yeast genomic DNA was then used in a PCR to amplify a 580-base pair (bp) product, Rpf2. Amplification of the 580 bp product from sample 1 (20 μg) and sample 2 (100 μg) demonstrates that the purified yeast genomic DNA was of high quality, pure, and suitable for PCR. See FIG. 10B.

Yeast genomic DNA was purified using the method of purifying DNA, RNA, and polypeptides disclosed herein. Yeast genomic DNA was also purified using a PROMEGA WIZARD® genomic DNA purification kit (available from Promega, Wisconsin, USA). The yeast genomic DNA purified using the method disclosed herein and the yeast genomic DNA purified using a PROMEGA WIZARD® genomic DNA purification kit were analyzed using WGS. Results in FIG. 10C show that the yeast genomic DNA purified using the method disclosed herein was of high quality, pure, and suitable for WGS. The results also show that the yeast genomic DNA purified using the method described herein was of greater quality compared to the yeast genomic DNA purified using a PROMEGA WIZARD® genomic DNA purification kit. See FIG. 10C.

The experiments in FIGS. 10A, 10B, and 10C demonstrate that multi-omic method of purifying DNA, RNA, and polypeptides disclosed herein is capable of isolating highly purified DNA in very high yield for whole genome sequencing.

### Example 11—Mass Spectrometry Analysis of a HEK Cell Proteome

Four separate human embryonic kidney (HEK) cell lysate samples (A, B, C, and D) were prepared and mixed with mTet-PEGₙ-CDM, thereby forming pH dependent tagged polypeptides, as described in Example 1. The mixed HEK cell lysates (including the pH dependent tagged polypeptides) were then added to TCO beads, as described in Example 1. After extensive washing, the proteins were eluted from the TCO beads by reversal of the polypeptide-CDM linkage, as described in Example 3. The eluted proteins were then digested, as described in Example 4, thereby forming peptides.

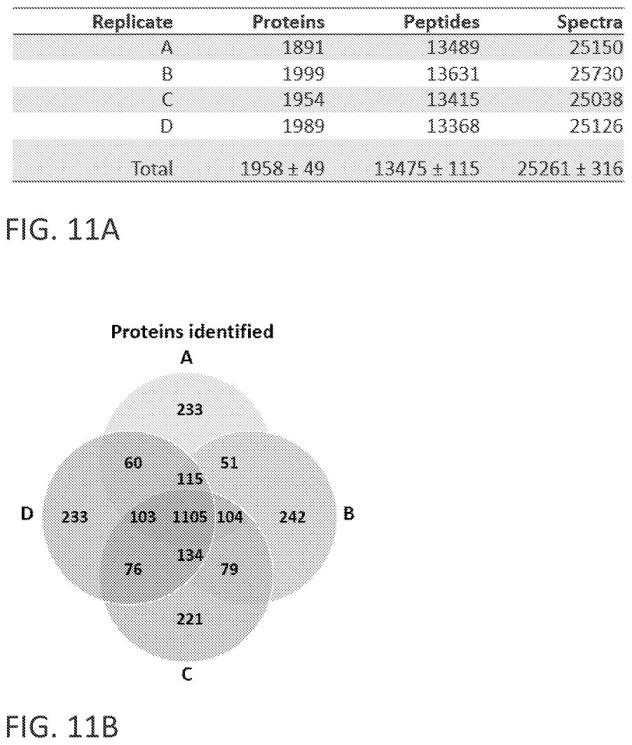

The peptides for each of sample A, B, C, and D were analyzed by Liquid-Chromatography-Mass Spectrometry (LC-MS/MS). See FIGS. 11A and 11B. In FIG. 11B, overlap of the proteins identified in each sample (A, B, C, D) is based on the p-value score.

These experiments in FIGS. 11A and 11B further demonstrate that the multi-omic method of purifying DNA, RNA, and polypeptides disclosed herein is highly repeatable in terms of protein capture, release, and analysis.

### Example 12—Analysis of Purified HEK Cell Genomic DNA

An experiment was performed to assess the quality of DNA recovered from the method of purifying DNA, RNA, and polypeptides disclosed herein.

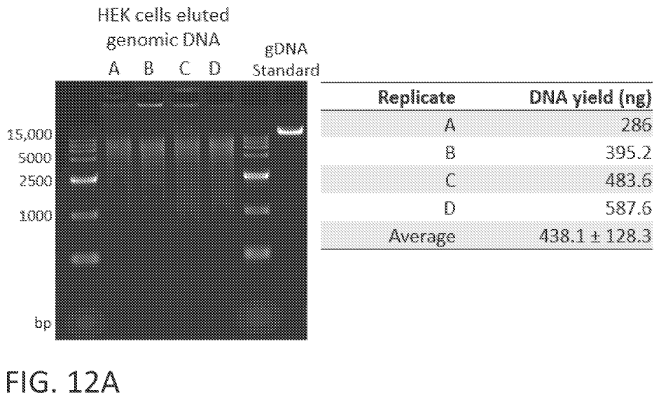

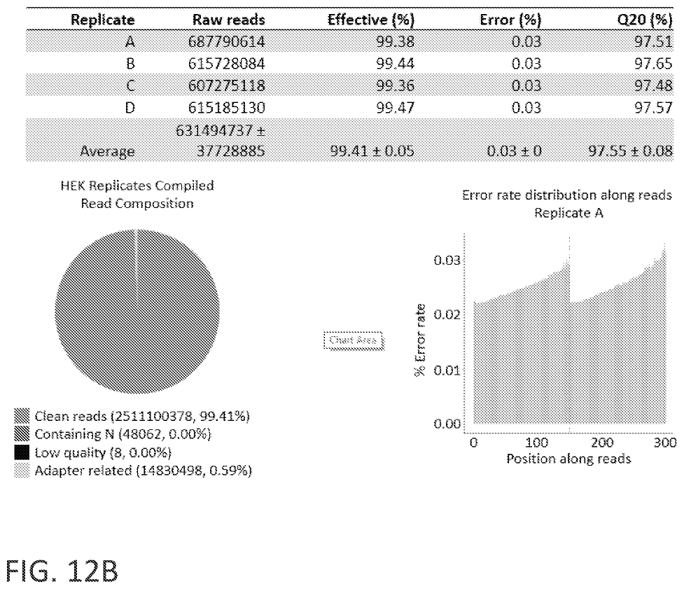

Four HEK cell lysates (A, B, C, and D) were prepared as described in Example 1. HEK cell lysates including 100 μg of protein were used to prepare purified DNA, as described in Example 2. The HEK cells eluted genomic DNA results are shown in FIG. 12A.

The HEK cells eluted genomic DNA (A, B, C, and D) was analyzed using WGS. Results in FIG. 12B show that the HEK cells genomic DNA purified using the method disclosed herein was of high quality, pure, and suitable for WGS. The results also show that 99.41% of the reads of the HEK cells genomic DNA were pure.

### Example 13—Analysis of Purified Cancer Cell Line RNA

An experiment was performed to assess the quality of RNA recovered from the method of purifying DNA, RNA, and polypeptides disclosed herein.

Cell lysates were prepared in triplicate from four different breast cancer cell lines (CA, T1, NeoT, and xA10) as described in Example 1. The breast cancer cell lines were at different stages of severity, ranging from least to most severe: xA10<NeoT<T1<CA1. Cell lysates including 100 μg of protein were used to prepare purified RNA, as described in Example 2.

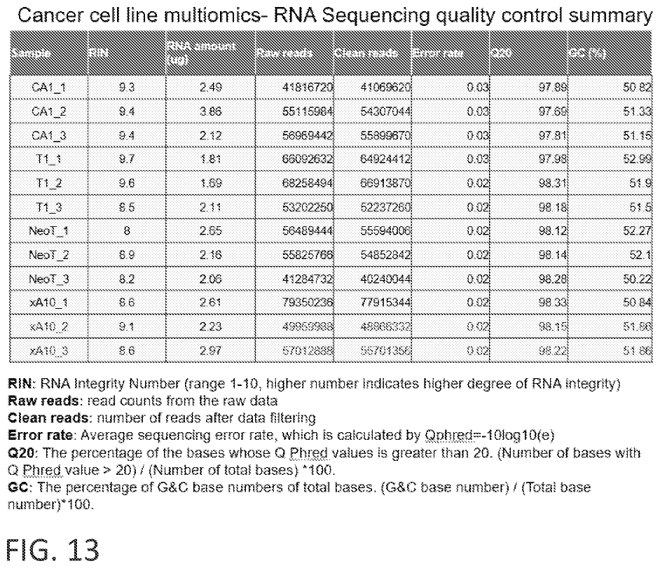

The breast cancer cells eluted RNA (CA, T1, NeoT, and xA10) was analyzed using RNA sequencing. Results in FIG. 13 show that the breast cancer cells RNA purified using the disclosed method was of high quality, high degree of RNA integrity, pure, and suitable for RNA sequencing.

### Example 14—Analysis of Purified Cancer Cell Line DNA

An experiment was performed to assess the quality of DNA recovered from the method of purifying DNA, RNA, and polypeptides disclosed herein.

Cell lysates were prepared in triplicate from four different breast cancer cell lines (CA, T1, NeoT, and xA10) as described in Example 1. Cell lysates including 100 μg of protein were used to prepare purified DNA, as described in Example 2.

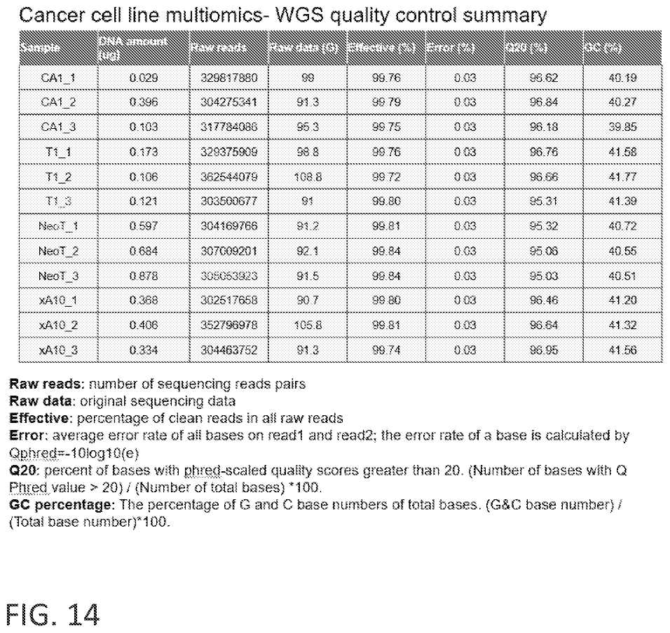

The breast cancer cells eluted DNA (CA, T1, NeoT, and xA10) was analyzed using WGS. Results in FIG. 14 show that the breast cancer cells DNA purified using the disclosed method was of high quality, pure, and suitable for WGS. The results also show that 99.76%, 99.79%, and 99.75% of the reads of the CA cancer cell line genomic DNA were pure; 99.76%, 99.72%, and 99.80% of the reads of the T1 cancer cell line genomic DNA were pure; 99.81%, 99.84%, and 99.84% of the reads of the NeoT cancer cell line genomic DNA were pure; and 99.80%, 99.81%, and 99.74% of the reads of the xA10 cancer cell line genomic DNA were pure. Again demonstrating the high quality of isolated DNA and the high repeatability of the multi-omic method of purifying DNA, RNA, and polypeptides disclosed herein.

### Example 15—Principle Component Analysis (PCA) of Cancer Cell Line Proteomes

Cell lysates were prepared in triplicate from four different breast cancer cell lines (CA, T1, NeoT, and xA10) and mixed with mTet-PEGₙ-CDM, thereby forming pH dependent tagged polypeptides, as described in Example 1. The mixed cancer cell lysates (including the pH dependent tagged polypeptides) were then added to TCO beads, as described in Example 1. After extensive washing, the proteins were eluted from the TCO beads by reversal of the polypeptide-CDM linkage, as described in Example 3. The eluted proteins were then digested, as described in Example 4, thereby forming peptides.

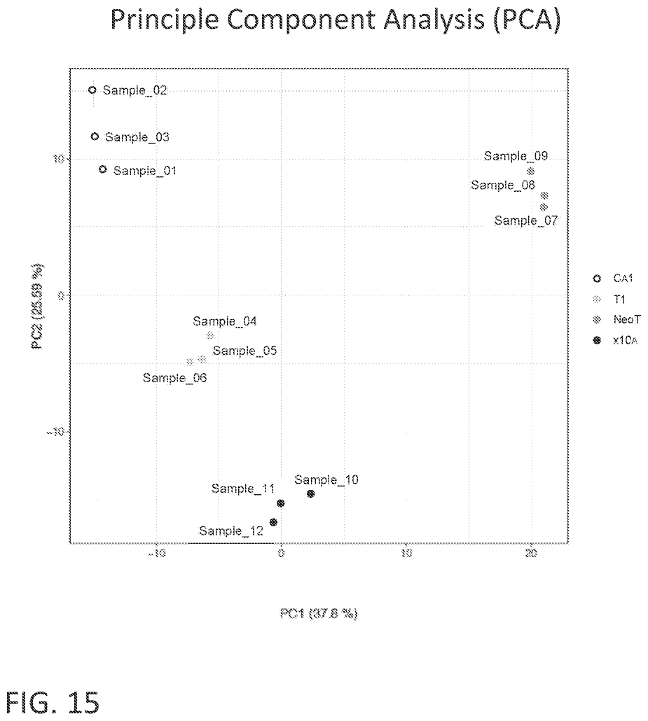

The peptides for each of breast cancer cell lines CA, T1, NeoT, and xA10 were analyzed by Liquid Chromatography-Mass Spectrometry (LC-MS/MS) (data not shown). The peptides for each of breast cancer cell lines CA, T1, NeoT, and xA10 were also analyzed by principle component analysis. See FIG. 15. These data show that the proteomes of these four breast cancer cell lines are indeed distinct and that the multi-omic method of purifying DNA, RNA, and polypeptides disclosed herein is highly reproducible.

While the present disclosure provides descriptions of various specific aspects for the purpose of illustrating various examples of the present disclosure and/or its potential applications, it is understood that variations and modifications will occur to those skilled in the art. Accordingly, the invention or inventions described herein should be understood to be at least as broad as they are claimed and not as more narrowly defined by particular illustrative examples provided herein.

1. A method of purifying DNA, RNA, and polypeptides from a sample, the method comprising:
a) mixing a sample comprising DNA, RNA, and polypeptides with a compound, thereby forming a mixed sample, wherein the compound comprises
a first moiety comprising a first member of a bio-orthogonal coupling pair, wherein the first member is configured to form a covalent bond with a second member of a bio-orthogonal coupling pair;
a second moiety configured to form a pH dependent covalent bond with a polypeptide; and
a linker linking the first moiety and the second moiety;
b) coupling polypeptides in the mixed sample to the second member of the bio-orthogonal coupling pair, wherein the second member of the bio-orthogonal coupling pair is linked to a substrate, thereby forming polypeptides bound to the substrate;
c) precipitating RNA and DNA in the mixed sample using a polar aprotic solvent, thereby forming precipitated RNA and precipitated DNA;
d) resolubilizing and eluting the precipitated RNA;
e) resolubilizing and eluting the precipitated DNA; and
f) eluting the polypeptides bound to the substrate in an elution buffer having a pH more acidic than a pH of the mixed sample by reversing the pH dependent covalent bond between the polypeptides and the second moiety.

2. A method of purifying DNA, RNA, and polypeptides from a sample, the method comprising:
a) coupling polypeptides in a mixed sample to a second member of a bio-orthogonal coupling pair, wherein the second member of the bio-orthogonal coupling pair is linked to a substrate, thereby forming polypeptides bound to the substrate, and wherein the mixed sample comprises DNA, RNA, polypeptides, and a compound comprising
a first moiety comprising a first member of a bio-orthogonal coupling pair, wherein the first member is configured to form a covalent bond with a second member of a bio-orthogonal coupling pair;
a second moiety configured to form a pH dependent covalent bond with a polypeptide; and
a linker linking the first moiety and the second moiety;
b) precipitating RNA and DNA in the mixed sample using a polar aprotic solvent, thereby forming precipitated RNA and precipitated DNA;
c) resolubilizing and eluting the precipitated RNA;
d) resolubilizing and eluting the precipitated DNA; and
e) eluting the polypeptides bound to the substrate in an elution buffer having a pH more acidic than a pH of the mixed sample by reversing the pH dependent covalent bond between the polypeptides and the second moiety.

3. The method of claim 1, further comprising:
mixing the polypeptides eluted in the elution buffer with a modified protease, thereby forming a mixed sample comprising peptides and the modified protease, wherein the modified protease comprises
a protease; and
a first member of a bio-orthogonal coupling pair attached to the protease, wherein the first member is configured to form a covalent bond with a second member of a bio-orthogonal coupling pair;
coupling the modified protease in the mixed sample to a second member of a bio-orthogonal coupling pair, wherein the second member of the bio-orthogonal coupling pair is linked to the substrate; and
eluting the peptides.

4. The method of claim 3, wherein the modified protease is
(i) a modified hydrolase and the protease is a hydrolase;
(ii) a modified serine hydrolase and the protease is a serine hydrolase; or
(iii) a modified trypsin and the protease is a trypsin.

5-6. (canceled)

7. The method of claim 1, wherein the second moiety is a dicarboxylic acid anhydride moiety.

8. The method of claim 7, wherein the dicarboxylic acid anhydride moiety is
(i) a maleic anhydride moiety; or
(ii) a 2-(2′-carboxyethyl) maleic anhydride moiety.

9. (canceled)

10. The method of claim 1, wherein the first member of the bio-orthogonal coupling pair is an electron-poor diene, an electron-rich dienophile, or a strained cycloalkene.

11. The method of claim 1, wherein the first member of a bio-orthogonal coupling pair is a tetrazine moiety selected from the group consisting of a 1,2,4,5-tetrazine moiety and a 4-(1,2,4,5-tetrazinyl)phenyl moiety.

12. The method of claim 1, wherein the linker is an inert linker.

13. The method of claim 1, wherein the substrate is
(i) a bead or a solid surface;
(ii) a magnetic bead;
(iii) a bead contained within a chromatography column or a spin column; or
(iv) a porous matrix.

14-16. (canceled)

17. The method of claim 1, wherein the sample comprising DNA, RNA, and polypeptides is a cell or tissue lysate having a pH greater than 7.

18. The method of claim 17, wherein the cell or tissue lysate is
(i) prepared in a solution comprising guanidinium thiocyanate;
(ii) prepared by mechanical homogenization; or
(iii) prepared in a solution comprising guanidinium thiocyanate and prepared by mechanical homogenization.

19. (canceled)

20. The method of claim 1, further comprising washing the polypeptides bound to the substrate using a polar aprotic solvent to remove any unbound materials from the polypeptides bound to the substrate.

21. The method of claim 1, wherein the polar aprotic solvent comprises acetonitrile or ethanol.

22. The method of claim 1, wherein the resolubilizing and eluting the precipitated RNA is achieved using
(i) an elution buffer comprising ultrapure water free of DNase and free of RNase;
(ii) an elution buffer having a pH greater than 7; or
(iii) an elution buffer comprising ultrapure water free of DNase and free of RNase and having a pH greater than 7.

23. (canceled)

24. The method of claim 17, wherein the pH greater than 7 is from greater than 7 to 10, greater than 7 to 9, or 8 to 9.5.

25. The method of claim 1, wherein the elution buffer having a pH more acidic than a pH of the mixed sample comprises a weak organic acid selected from the group consisting of formic acid, acetic acid, and citric acid.

26. The method of claim 1, wherein the pH more acidic than a pH of the mixed sample is from 2 to less than 7, 3 to less than 7, or from 2.5 to 6.

27. A method of purifying DNA, RNA, and polypeptides from a sample, the method comprising:
a) mixing a sample comprising DNA, RNA, and polypeptides with a compound, thereby forming a mixed sample, wherein the compound comprises
a first moiety comprising a first member of a bio-orthogonal coupling pair;
a second moiety comprising dicarboxylic acid anhydride; and
a linker linking the first moiety and the second moiety;
b) coupling polypeptides in the mixed sample to a second member of a bio-orthogonal coupling pair, wherein the second member of the bio-orthogonal coupling pair is linked to a substrate, thereby forming polypeptides bound to the substrate;
c) precipitating RNA and DNA in the mixed sample using a polar aprotic solvent, thereby forming precipitated RNA and precipitated DNA;
d) resolubilizing and eluting the precipitated RNA;
e) resolubilizing and eluting the precipitated DNA; and
f) eluting the polypeptides bound to the substrate in an elution buffer having a pH more acidic than a pH of the mixed sample by reversing a pH dependent covalent bond between the polypeptides and the second moiety.

28-50. (canceled)

51. A multi-omic kit configured to perform the method of claim 1.

52-76. (canceled)
